(19) United States
(12) Patent Application Publication
(10) Pub. No.: US 2008/0120732 A1
(43) Pub. Date: May 22, 2008

(54) Method for Improved Transgene Expression

(75) Inventor: Elizabeth Elliot, Midlothian (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Viragen Inc., Plantation, FL (US)

(21) Appl. No.: 11/661,771

(22) PCT Filed: Sep. 2, 2005
(86) PCT No.: PCT/GB05/03402

§ 371 Date: Aug. 13, 2007

(30) Foreign Application Priority Data
Sep. 2, 2004 (GB) 0419424.7

(51) Int. Cl. C12P 21/00; C12N 15/87; C12N 15/00; C12N 5/00; C07H 21/04

(52) U.S. Cl. 800/4; 435/463; 536/22.1; 800/25; 435/320.1; 435/325

(57) ABSTRACT

The present invention provides an improved method for achieving efficient transcription and translation of modified transgene constructs in vector systems. The vector may be a lentiviral vector. Such a method facilitates the production of viral vector genomes with intact functional transgene sequences allowing stable integration of a transgene-containing viral vector genome into the germline of an animal such as a transgenic avian. The subsequent expression of the transgene results in a recombinant protein product being produced, which, in the case of a transgenic avian can result in the targeted production of the protein into the egg of the transgenic bird.

### FIELD OF INVENTION

The present invention provides an improved method for achieving efficient transcription and translation of modified transgene constructs in vector systems, and in particular lentiviral vectors. Such a method facilitates the production of viral vector genomes with intact functional transgene sequences allowing stable integration of a transgene-containing viral vector genome into the germline of an animal such as a transgenic avian. The subsequent expression of the transgene results in a recombinant protein product being produced, which, in the case of a transgenic avian can result in the targeted production of the protein into the egg of the transgenic bird.

### BACKGROUND TO THE INVENTION

Traditional methods for the manufacture of recombinant proteins include production in bacterial or mammalian cells. An alternative manufacturing approach uses transgenic animals and plants for the production of proteins.

A number of protein-based biopharmaceuticals have been expressed in the milk of a range of mammals such as transgenic mice, rabbits, pigs, sheep, goats and cows. Such systems tend to have long generation times, with the larger mammals taking years to develop from the founder transgenic to a stage at which they can produce milk.

Additional difficulties relate to the biochemical complexity of milk and the evolutionary conservation between humans and mammals, which can result in adverse reactions to the pharmaceutical in the mammals which are producing it (Harvey et al., 2002).

There is increasing interest in the use of chicken eggs as a potential manufacturing vehicle for pharmaceutically important proteins, especially recombinant human antibodies.

A protein manufacturing system based on chicken eggs has several advantages as compared to mammalian cell culture, or the use of transgenic mammalian systems. Chickens have a short generation time (24 weeks), which permits transgenic flocks to be established rapidly. Secondly, the capital outlays for a transgenic animal production facility are far lower than that for cell culture. Extra processing equipment required to facilitate transgenic protein production is minimal in comparison to that required for cell culture. These lower capital outlays result in the production cost per unit of transgenic therapeutic being lower than that produced by cell culture. In addition, transgenic systems provide significantly greater flexibility regarding purification batch size and frequency. This flexibility may lead to further reductions in capital and operating costs in purification through batch size optimisation.

Further, transgenic protein production results in increased speed to market. Transgenic mammals are capable of producing several grams of protein product per litre of milk, making large-scale production commercially viable (Weck, 1999). Further, the short generation time for birds allows a rapid scale up of production.

The avian egg, and in particular the egg of the chicken, offers several major advantages over cell culture as a means of protein production. Further, the avian system provides significant advantages over other transgenic production systems based upon mammals or plants.

Direct application of the methods used in the production of transgenic mammals to the genetic manipulation of birds has not been possible because of specific features of the reproductive system of the laying hen.

The complexities of egg formation make the earliest stages of chick-embryo development relatively inaccessible. Methods employed to access earlier stage embryos usually involve sacrificing the donor hen to obtain the embryo or direct injection into the oviduct. Methods for the production of transgenic mammals have focused almost exclusively on the microinjection of a fertilised egg, whereby a pronucleus is microinjected in-vitro with DNA and the manipulated eggs are transferred to a surrogate mother for development to term, this method is not feasible in hens.

Four general methods for the creation of transgenic avians have been developed. These are (i) a method for the production of transgenic chickens using DNA microinjection into the cytoplasm of the germinal disk, (ii) the transfection of primordial germ cells in-vitro and transplantation into a suitably prepared recipient, (iii) the use of gene transfer vectors derived from oncogenic retroviruses, and (iv) the culture of chick embryo cells in-vitro followed by production of chimeric birds by introduction of these cultured cells into recipient embryos (Pain et al., 1996). The embryo cells may be genetically modified in-vitro before chimera production, resulting in chimeric transgenic birds.

Lentiviruses are a subgroup of the retroviruses which include a variety of primate viruses such as human immunodeficiency viruses HIV-1 and HIV-2, simian immunodeficiency virus (SIV) and non-primate viruses (e.g. maedi-visna virus (MVV), feline immunodeficiency virus (FIV), equine infectious anaemia virus (EIAV), caprine arthritis encephalitis virus (CAEV) and bovine immunodeficiency virus (BIV)). These viruses are of particular interest in development of gene therapy treatments, since not only do the lentiviruses possess the general retroviral characteristics of irreversible integration into the host cell DNA, but they also have the ability to infect non-proliferating cells. The biology of lentiviral infection can be reviewed in Coffin et al., (1997).

An important consideration in the design of a viral vector is the ability to be able to stably integrate into the genome of cells. Previous work has shown that oncoretroviral vectors used as gene transfer vehicles have had somewhat limited success due to the gene silencing effects during development. The work of Pfeifer et al., (2002) and Lois et al., (2002) on mice has shown that a lentiviral vector based on HIV-1 is not silenced during development.

The bulk of the developmental work on lentiviral vectors has been focused upon HIV-1 systems, largely due to the fact that HIV, by virtue of its pathogenicity in humans, is the most fully characterised of the lentiviruses. Such vectors tend to be engineered so as to be replication incompetent, through removal of the regulatory and accessory genes, which render them unable to replicate. The most advanced of these vectors have been minimised to such a degree that almost all of the regulatory genes and all of the accessory genes have been removed.

The lentiviral group of viruses have many similar characteristics, such as a similar genome organisation, a similar replication cycle and the ability to infect mature macrophages (Clements & Payne, 1994). One such lentivirus is Equine Infectious Anaemia Virus (EIAV). Compared with the other viruses of the lentiviral group, EIAV has a relatively simple genome: in addition to the retroviral gag, pol and env genes, the genome only consists of three regulatory/accessory genes (tat, rev and S2). The development of a safe and efficient lentiviral vector system will be dependent on the design of the vector itself. In order to obtain effective function, it is important to minimise the viral components of the vector, whilst still retaining its transducing vector function.

Oncoretroviral and lentiviral vectors systems may be modified to broaden the range of transducible cell types and species. This is achieved by substituting the envelope glycoprotein of the virus with other virus envelope proteins.

It is possible to achieve stable germline expression of a transgene packaged into EIAV lentiviral vectors (McGrew et al., 2004). This method involves the synthesis of the relevant piece of exogenous DNA and alteration of the codon usage for the optimal chicken frequencies observed (a process colloquially referred to as ‘chickenisation’). This process may be sufficient to enable efficient transcription and translation of certain exogenous DNA sequences, resulting in expression of the protein in the resultant bird. However, it has been shown that some protein sequences require modification in order to be able to be stably expressed.

The murine antibody known as R24, specific for the ganglioside GD3, was used to create a recombinant antibody-like binding molecule termed a ‘minibody’. The minibody structure comprised traditional antibody V_{H} and V_{L} domains joined by a linker and the Fc domain of IgG1. The coding sequence for this minibody was packaged into an EIAV-based lentivector, however subsequent expression of the minibody protein product could not be achieved.

Sequence analysis of RT-PCR products amplified directly from various R24 minibody-containing viral genomes identified the occurrence of numerous deletions encompassing some or all of the exogenous R24 minibody coding sequence. An analysis of the sequence delineating the 5′ and 3′ extent of these deletions, indicated that aberrant splicing is not responsible for these deletions. The deletions appear to be defined by small (5-10 bp) direct repeats, this suggesting that a previously unknown homologous recombination-based mechanism is responsible for the changes to the exogenous DNA coding sequence seen.

Ch'ang et al. have previously reported internal deletions in integrated proviral genomes of murine leukemia virus (MuLV) stating that all three of the deletions identified during the study were flanked by 7 nucleotide direct repeats (Ch'ang et al, 1989). Specific deletions involving DNA sequences flanked by short direct repeats have also been observed in other retroviral genes (reviewed by Coffin, 1985) and in various prokaryotic and eukaryotic genes (discussed in Omer et al., 1983 and Levy et al., 1985). Deletions flanked by short direct repeats have also been observed in the avian sarcoma virus src gene (Omer et al., 1983). It is suggested that the proposed mechanism is slippage of DNA replicative machinery, for example DNA polymerase or reverse transcriptase. However, the deletions observed in the R24 minibody vector system were in RT-PCR products amplified directly from reverse transcribed viral RNA genomes and as such they cannot be explained by this mechanism. Instead it is more probable that the host cell RNA polymerase (Rpol II) introduced deletions during the transcription of the viral genomes immediately after the transfection of the plasmid into the packaging cell line. In support of this conclusion it is known that some host DNA-dependent RNA polymerases are capable of template switching (Nudler et al., 1996) and that RNA recombination is affected by the presence of 3D structure such as hairpin loops (White & Morris, 1995).

Another exogenous gene sequence, that of the recombinant murine anti-CD55 antibody known as 791T/36, was assessed for predisposition for deletion occurrence when incorporated into a lentiviral vector backbone. Sequences known to be involved in deletions were conserved in 791T/36.

It is therefore possible that certain sequences within genes encoding some complex proteins may be predisposed to experience deletion when incorporated into the lentiviral vector backbone. It is likely that the extent of any deletion(s) will differ dramatically from gene to gene and therefore would be unpredictable. As has been demonstrated in relation to the expression of the R24 minibody, deletions may occur to such an extent that protein expression is no longer possible from the transgene, which in turn prevents the expression of the protein in the transgenic system.

It would be highly desirable to be able to screen exogenous DNA sequences prior to their inclusion in an expression vector in order to identify areas of sequence which may have a predisposition for deletion.

The inventors of the present invention have surprisingly developed a screening method which allows exogenous DNA sequences to be analysed to determine areas of sequence where a predisposition to deletion or other forms of sequence modification may exist. Once identified, such areas of sequence can be modified. Further, such modification can be advantageously performed prior to the inclusion of the exogenous DNA sequence into a vector backbone. This method therefore facilitates the production of viral vector genomes with intact functional transgene sequences allowing stable integration of a transgene-containing viral vector genome into the germline of an animal such as a transgenic avian and as such can be used in the production of recombinant proteins in transgenic systems such as non-human animals and in particular in avians.

### SUMMARY OF THE INVENTION

According to a first aspect of the present invention there is provided a method of optimising an exogenous DNA sequence for expression by a suitable vector, the method comprising at least one of the steps of:
(i) optimising the nucleotide codon usage of the exogenous DNA to alter codon usage to that of the host cell type in which the exogenous DNA sequence is to be expressed,
(ii) modifying the codon optimised exogenous DNA sequence to alter any area of sequence which may prevent or down regulate expression of the exogenous DNA in the host cell, and
(iii) altering the nucleotide codon usage of the exogenous DNA sequence in order to remove all sequences implicated in the putative homologous recombination-based deletion mechanism.

In one embodiment, the method comprises steps (i) and (iii). In a further embodiment, the method comprises steps (ii) and (iii). In a yet further embodiment, the method comprises steps (i), (ii) and (iii).

Sequence elements which are predicted to prevent or down regulate expression of the coding sequence in the host cell may include; negative elements or repeat sequences, cis-acting motifs such as splice sites, internal TATA-boxes or ribosomal entry sites.

Accordingly, embodiments of the invention extend to analysing the exogenous DNA sequence for the presence of any sequence elements which may prevent or down regulate expression of the exogenous DNA in the host cell selected, in particular said sequence elements may be selected from the group comprising; negative elements or repeat sequences, cis-acting motifs such as splice sites, internal TATA-boxes and ribosomal entry sites.

Such negative elements commonly fit within one of two categories; for example generic sequences such as those that are AT or GC rich or would be predicted to contribute to significant RNA secondary structure or, defined consensus sequences to which specific functions have been attributed such an internal TATA box, chi site, ribosomal entry site, ARE, INS, CRS, splice signals or polyadenylation signal.

A TATA box can be defined as a consensus sequence found in the promoter region of most genes transcribed by eukaryotic RNA polymerase II which is located around 25 nucleotides before the site of initiation of transcription (5′ TATAAAA 3′). The sequence seems to be important in determining accurately the position at which transcription is initiated.

RecBCD enzyme is a heterotrimeric helicase/nuclease that initiates homologous recombination at double-stranded DNA breaks. Several of its activities are regulated by the DNA sequence chi (5′ GCTGGTGG 3′) which is recognised in cis by the translocating enzyme (Spies et al, 2003).

Internal ribosomal entry sites are usually defined on a functional basis and those so far reported do not share significant sequence homology. However an in silico sequence analysis programme can verify that no known IRES sequences are present within the transgene sequence (reviewed in Martinez-Salas, 1999).

Adenine Rich Elements (AREs) are defined as AU-rich sequence frequently located in the 3′UTR of mRNAs from transiently expressed genes. The introduction of an ARE sequence is sufficient to confer instability on mRNAs and as such they have been proposed to be a recognition signal for an mRNA processing pathway (Shaw & Kamen, 1986).

Inhibitory Sequences (INS) and Cis-acting Repressor, Sequences (CRS) were both initially reported in an HIV model system and one hypothesis is that they are binding sites for cellular factors which contribute to mRNA instability (Schneider et al, 1997). It has been demonstrated that the removal of such sequences from HIV transcripts results in a significant boost in the expression of those transcripts (Schneider et al, 1997) and as such the verification of the absence or removal of, previously defined INS or CRS sequences is desirable during the transgene optimization process.

Three types of consensus splice signals have been documented. First the splice donor (C or A, A, G/G T, A or G, A, G, T that defines the 5′ end of the sequence to be excised, the “intron”. Second the splice acceptor (T or C, n, N, C or T, A, G/g that defines the 3′ extent of the sequence to be excised. Third the branch point sequence (TACTAAC) located within the sequence to be excised and is involved in lariat formation during the splicing reaction.

Termination of transcription by RNA polymerase II usually requires the presence of a functional polyadenylation signal (poly(A)). The core poly(A) signal in vertebrates consists of two recognition elements flanking a cleavage poly(A) site. Typically, an almost invariant AAUAA hexamer lies 20 to 50 nucleotides upstream of a more variable element rich in U or GU residues. Cleavage of the nascent transcript occurs between these two elements and is coupled to the addition of up to 250 adenosines, the poly(A) tail, to the 5′ cleavage product (Tran et al, 2001).

The consequences of retaining some or all of the above sequence elements will vary depending on the nature of the retained sequence. They are broadly described as negative elements as all conspire to reduce expression of the heterologous coding sequence although by a variety of different mechanisms. For example, the retention of cognate splicing sequences within a heterologous coding sequence would result in high efficiency splicing and deletion which depending on the location could abolish, reduce or permit expression of a truncated gene product. In contrast retention of an INS element would not affect RNA integrity, rather the mRNA would be targeted for rapid degradation before significant translation of the desired encoded gene product could occur. Both mechanisms yield the same general outcome, a reduction in the levels of heterologous protein expression.

In one embodiment of this aspect of the invention, the exogenous DNA sequence which has been analysed and optionally modified according to the method for optimising expression of the invention is included in a vector which may be expressed in a transgenic expression system.

The transgenic expression system may be a non-human mammal. In a yet further embodiment the transgenic expression system may be an avian, in particular a chicken or quail.

In one embodiment of the invention, the exogenous DNA encodes for a heterologous protein which is placed under the control of an internal promoter of the vector and which will be expressed by the host cell.

In one embodiment the vector is a lentiviral vector. In a further embodiment the vector is Equine Infectious Anaemia Virus (EIAV). The invention also provides for the lentiviral vector to be human immunodeficiency viruses HIV-1 and HIV-2, simian immunodeficiency virus (SIV), non-primate viruses for example maedi-visna virus (MVV), feline immunodeficiency virus (FIV), equine infectious anaemia virus (EIAV), caprine arthritis encephalitis virus (CAEV) and bovine immunodeficiency virus (BIV)).

In an embodiment of this aspect of the invention, the exogenous DNA may encode for a heterologous protein being a recombinant antibody or other similar binding fragments or members.

Analysis of an exogenous DNA sequence encoding for such an antibody or binding member may additionally include the step of designing a linker sequence for inclusion in the antibody or binding member which has all direct repeats removed from the DNA sequence, while still retaining the three direct repeats of (Gly₄Ser₁) in the primary amino acid sequence. This step is preferably performed prior to the performance of step (iii) when performed as part of the method according to this aspect of the invention.

More specifically, such a step would be performed following the completion of step (ii) and prior to the performance of step (iii), this step therefore being herein referred to as step (iib) of the method of this aspect of the present invention.

As herein defined, the term ‘codon optimisation’ refers to the process of altering codon usage such that the codon usage of the exogenous DNA sequence is deliberately biased to encode for those codons most frequently used in the non-human mammal host cell type into which the vector is to be inserted and expressed in order to improve expression. For example, where the transgenic expression system is a chicken, the alteration of codon usage will change certain codons in order to bias their expression towards those most commonly used in the chicken species. When performed in chickens, this step of altering codon usage of the nucleotide sequence may be colloquially referred to as the process of ‘chickenising’ or ‘chickenisation’ of the exogenous DNA sequence.

More particularly, as herein defined, the term ‘chickenisation’ refers to the process of deliberately altering codon usage in a nucleotide sequence such that a codon is encoded by the 3 nucleotides which are most prevalent in the chicken species for encoding the amino acid which is encoded by the nucleotide sequence (codon) in its unaltered form. For expression in transgenic chickens the codons formed by the exogenous DNA sequence are optimised to the most frequent codon usage pattern in chickens. However, it can be seen that the optimisation could be for the most frequent codon usage of any avian species, or non-human mammal in which the vector is expressed.

For an example of how chickenisation is carried out, it can be seen that the amino acid valine is encoded by 4 different codons, GTG, GTA, GTT and GTC with GTG being used most frequently in chickens (46% GTG, 11% GTA, 19% GTT and 23% GTC). To chickenise the human IgG Fc DNA, all valine codons were converted to GTG. Lysine is encoded by two different codons, AAG and AAA, with AAG used most frequently in chickens (58% vs 42%). All AAA codons in the sequence were converted to AAG. Not all codons required alteration. For example, the two codons for aspartic acid, GAT and GAC are used almost equally (48% vs. 52%) and hence are not required to be changed during the chickenisation procedure.

The steps of altering codon usage and sequence modification as outlined in steps (i) and (ii) of the method of this aspect of the present invention are known to those skilled in the art for the optimisation of gene expression from heterologous transgenes (see for example, Graf et al., 2000).

Steps (i) and (ii) of the method of this aspect of the present invention may be typically performed in collaboration with Geneart GmbH (Germany, www.geneart.com) or organisations which provide similar sequence design services. The performance of steps (i) and (ii) by Geneart typically comprise the performance of computer assisted sequence design which allows sequence design and analysis in order to achieve sequence optimisation. This process includes the steps of analysing a sequence and swapping codon usage and then analysing the resulting sequence in order to ensure that the sequence changes resulting from the codon swapping do not introduce any negative elements or repeats. A more specific description of the method of optimising the nucleotide sequence for expression of a protein can be found in International PCT Patent Application No WO 2004/059556, the contents of which are incorporated herein by reference.

The resulting base sequence is then further modified as defined in step (iii). Optionally, an additional step, termed (iib), as defined above, can be performed prior to the performance of step (iii).

The final sequence may then be re-analysed to ensure no problematic sequences have been reintroduced before synthesis of the exogenous DNA sequence is initiated.

It can be seen that this process can be adapted for use with any protein sequence as necessary, by simply adapting steps (iib) and (iii) to utilise the appropriate sequences, depending on the exogenous DNA sequence to be expressed.

The modular nature of the screening method makes it highly adaptable in that it may be applied to any exogenous DNA sequence that may be at risk of deletion occurrence following its integration into a vector, such as a lentiviral vector, when used for the creation of a transgenic animal. For example, the coding sequence of a standard transgene, such as an enzyme or a bioactive protein such as a cytokine or hormone may be analysed, as may the sequence of any other protein, such as a therapeutic protein, the expression of which is desirable in a non-human mammalian transgenic system.

Furthermore, the screening method may be used to screen the sequence of an antibody or other similar binding fragment or member.

An “antibody” is an immunoglobulin, whether natural or partly or wholly synthetically produced. The term also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd, and diabodies. The antibody may be humanised and this may include antibodies which are partly humanised (chimaeric) or fully humanised.

However, if the screening method of this aspect of the invention is to be used for the optimisation of expression of recombinant antibody-based transgenes it is recommended that a modified linker sequence be used.

### Linker Sequence Development

An example of a widely used commercially available linker which is found in the RPAS Mouse scFV Module (Amersham Biosciences), the linker sequence has a nucleotide sequence as shown below as SEQ ID NO 1:

GGT GGA GGC GGT TCA GGC GGA GGT GGC TCT GGC GGT
GGC GGA TCG

The nucleotide sequence of SEQ ID NO 1 encodes for an amino acid sequence having the sequence of SEQ ID NO 2:

Gly Gly Gly Gly Ser Gly Gly Gly Gly Ser Gly Gly
Gly Gly Ser

The present invention additionally provides a new linker which has been designed and which has the nucleotide sequence as follows as SEQ ID NO 3;

GGG GGA GGG GGC AGC GGC GGA GGG GGA TCC GGC GGT
GGG GGA TCT

The nucleotide sequence of SEQ ID NO 3 encodes for an amino acid sequence having the sequence of SEQ ID NO 4:

Gly Gly Gly Gly Ser Gly Gly Gly Gly Ser Gly Gly
Gly Gly Ser

As well as being designed to exclude the presence of repeat DNA sequences, a second constraint applied during sequence design and analysis of the linker sequence was the avoidance of GGC and TCC as adjacent codons. For example, when the widely-used commercially available linker which is found in the RPAS Mouse scFV Module (Amersham Biosciences) (SEQ ID NO 5) is assessed for the presence of GGC and TCC as adjacent codons, the following is observed:

### SEQ ID NO 5:

GGG GGA GGC GGC TCC GGG GGA GGC GGC TCC GGG GGA
GGC GGC TCC

The re-design process was carried out since previous PCR data from several EIAV based lentiviral vector constructs, known as pRI28 (CMV promoter driving R24 minibody expression) and pLE38 (a tissue specific promoter driving R24 minibody expression) have implicated this repeat in a putative homologous recombination-based mechanism causing deletions in the R24 minibody coding sequence. The new linker also avoids the use of so-called “slow pairs” of codons, GGA GGC (Trinh et al., 2004) which are known to cause poor expression levels of recombinant proteins that contain them.

The use of a non-repetitive linker sequence is known in the art. However, the present invention further provides for the modification of the exogenous DNA sequence to modify codon selection within the linker to remove short, direct repeat elements from viral vector transgenes.

A yet further aspect of the present invention provides isolated DNA which encodes at least part of a heterologous protein, said DNA having been analysed in accordance with the screening method of the present invention.

A yet further aspect of the present invention provides a linker sequence for the expression of a recombinant antibody-based transgene, said linker sequence having a nucleotide sequence according to SEQ ID NO 3.

A yet further aspect of the present invention provides a linker sequence for the expression of a recombinant antibody-based transgene, said linker sequence having a nucleotide sequence according to SEQ ID NO 4.

A further aspect of the present invention provides a method of producing a transgenic avian, the method comprising the steps of;
providing an exogenous DNA sequence which encodes for at least one heterologous protein, the expression of which is desired in the transgenic avian,
performing codon optimisation of the nucleotide sequence of the heterologous protein coding region of the exogenous DNA sequence to alter codon usage to that of the avian cell in which the heterologous protein is to be expressed,
modifying the exogenous DNA sequence to alter any coding sequence regions which are predicted to prevent or down regulate gene expression in the host avian,
altering codon usage of the exogenous DNA sequence in order to remove all sequences implicated in the putative homologous recombination-based deletion mechanism,
integrating a vector comprising the exogenous DNA sequence into the genome of an avian, and
expressing said coding sequence in order to produce the heterologous protein encoded by said sequence.

In preparing a vector which comprises the exogenous DNA sequence of the invention, the exogenous DNA sequence will be packaged along with associated regulatory and expression control regions. The skilled person will be aware of suitable methods for packaging the vector.

The invention thus also provides a transgenic avian. A transgenic avian is any member of the avian species, in particular the chicken, wherein at least one of the cells of the avian contains, integrated within that cell's genome, the exogenous genetic material contained in the vector. Transgenic techniques which are suitable for the introduction of such genetic material will be known to the person skilled in the art.

The methods of the present invention can be used to generate any transgenic avian, including but not limited to chickens, turkeys, ducks, quail, geese, ostriches, pheasants, peafowl, guinea fowl, pigeons, swans, bantams and penguins. Chickens are however preferred.

The heterologous protein expressed by the transgenic avian may be, but is not limited to proteins having a variety of uses including therapeutic and diagnostic applications for human and/or veterinary purposes and may include sequences encoding antibodies, antibody fragments, antibody derivatives, single chain antibody fragments, fusion proteins, peptides, cytokines, chemokines, hormones, growth factors or any recombinant protein.

The present invention further extends to a chimeric avian or a mosaic avian, wherein the exogenous genetic material is found in some, but not all of the cells of the avian.

In one embodiment the transgenic avian expresses the exogenous genetic material in the oviduct so that the expressed genetic material, in the form of a translated protein, becomes incorporated into the egg.

A lentiviral vector expression construct may be used to direct expression of a heterologous protein encoded by the vector to specific tissues (tissue-specific expression). In one embodiment, such tissue specific expression is directed such that this results in the inclusion of the heterologous protein in the egg. This may be in the egg white or egg yolk, however it is preferable that the protein is present in the egg white.

The protein can then be isolated from the egg white or yolk by standard methods which will be known to the person skilled in the art.

A yet further aspect of the present invention provides a method of expressing at least one heterologous protein in the oviduct of an avian, the method comprising the steps of;
providing an exogenous DNA sequence which has been analysed using the method of the present invention in order to remove or replace any areas of coding sequence which may prevent or down regulate the expression of the heterologous protein encoded by the exogenous DNA sequence,
integrating a vector comprising the exogenous DNA coding sequence into the genome of an avian,
expressing the exogenous DNA coding sequence by means of a promoter which is operably linked to the exogenous DNA sequence, and
obtaining the exogenous protein expressed by said transgenic avian.

In one embodiment the exogenous DNA coding sequence which has been analysed according to the screening method of the first aspect of the present invention is inserted into a viral vector backbone, with this vector being inserted into an avian cell.

It is preferred that the promoter effects ‘tissue specific’ expression of the heterologous protein encoded by the exogenous DNA sequence in the tubular gland cells of the magnum portion of the avian oviduct. ‘Tissue specific’ expression results in the expression of the heterologous protein to a specific tissue, with the exclusion of expression of the heterologous protein in other tissues. An example of a promoter which would be predicted to direct tissue specific expression of the heterologous protein to the oviduct of an avian would be the ovalbumin promoter.

In further embodiments of this aspect of the invention, the promoter may be altered as required, in order to direct expression of the heterologous protein encoded by the exogenous DNA coding sequence to other tissues of the avian.

The exogenous protein may be a therapeutically useful protein. In particular the heterologous protein expressed may be an antibody or similar binding fragment or member.

A yet further aspect of the present invention provides a method of expressing at least one exogenous protein in an avian, said method comprising the steps of:
providing an exogenous DNA sequence encoding for an exogenous protein which is to be expressed,
analysing said exogenous DNA sequence using the screening method according to the present invention,
expressing the exogenous DNA sequence into the genome of an avian,
obtaining the expressed antibody protein from the avian.

In one embodiment of this aspect of the invention, the at least one heterologous protein is expressed in a tissue specific manner, most preferably, in the oviduct of the avian, by virtue of tissue specific expression in the cells of the oviduct. In another embodiment, the exogenous protein is expressed in the tubular gland cells of the magnum portion of an avian oviduct, with the exogenous protein being deposited in the white of an egg. Alternatively, or in addition, the heterologous protein may be deposited in the egg yolk or secreted into the blood.

In a further embodiment the avian is a chicken.

In one embodiment the heterologous protein expressed in the oviduct is an antibody. In a further embodiment the antibody is ‘humanised’.

A further still aspect of the present invention provides for the use of an exogenous DNA sequence which has been analysed using the screening method of the first aspect of the present invention in the production of an avian egg containing an exogenous protein.

In one embodiment the exogenous protein is deposited within the egg white. In further embodiments, the exogenous protein is contained in the yolk of the egg.

A further still aspect of the present invention provides for the use of an exogenous DNA sequence which has been analysed with the screening method of the first aspect of the present invention in the production of a heterologous protein product, said protein product being the result of transcription and translation of at least part of the exogenous DNA sequence.

A further aspect of the present invention provides an expression vector which comprises at least one exogenous DNA sequence which has been analysed according to the screening method of the first aspect of the present invention.

A yet further aspect provides a host cell transduced with an expression vector as defined above.

In one embodiment the expression vector is a lentiviral expression vector, in particular EIAV.

In one embodiment the host cell is a non-human mammalian cell. In further embodiments, the host cell is an avian cell, in particular a chicken cell.

In a still further aspect of the present invention there is provided a kit for the performance of any one of the methods of the invention, said kit comprising instructions and protocols for the performance of said method(s).

Preferred features and embodiments of each aspect of the invention are as for each of the other aspects mutatis mutandis unless the context demands otherwise.

### DEFINITIONS

The terms “vector”, “viral vector” and “expression vector” are used interchangeably herein, and refer to any nucleic acid, preferably DNA, which allows for promoter induced expression, that is transcription and subsequent translation, of an exogenous DNA sequence.

The viral vector genome is preferably “replication defective”, that is that the genome of the vector does not comprise sufficient genetic information alone to allow independent replication to result in the production of infectious viral particles. In the case a of a lentiviral vector, the genome would lack a functional gag, env or pol gene.

The term “Lentivirus” refers to the family of retroviruses particularly preferred for the present invention. Lentiviruses include a variety of primate viruses such as human immunodeficiency viruses HIV-1 and HIV-2 and simian immunodeficiency virus (SIV) and non-primate viruses (e.g. maedi-visna virus (MVV), feline immunodeficiency virus (FIV), equine infectious anaemia virus (EIAV), caprine arthritis encephalitis virus (CAEV) and bovine immunodeficiency virus (BIV)).

“Viral vector genome” refers to a polynucleotide comprising sequences from a viral genome that is sufficient to allow an RNA version of that polynucleotide to be packaged into a viral particle, and for that packaged RNA polynucleotide to be reverse transcribed and integrated into a host cell chromosome. Heterologous sequences such as the promoter sequence and the exogenous DNA sequence which encodes for a heterologous peptide may also be part of the viral vector genome.

The term “recombinant”, as used herein to describe a nucleic acid molecule, means a polynucleotide of genomic, cDNA, semi-synthetic, or synthetic origin, which by virtue of its origin or manipulation is not associated with all or a portion of the polynucleotide with which it is associated in nature, and/or is linked to a polynucleotide other than that to which it is linked in nature.

The term “recombinant”, as used herein to describe a protein or polypeptide means a polypeptide produced by expression of a recombinant polynucleotide.

As used herein, the term “nucleic acid” includes DNA, RNA, mRNA, cDNA, genomic DNA, and analogues thereof.

A “exogenous DNA sequence” is a nucleic acid sequence for which transcriptional expression is desired. The exogenous DNA sequence will generally encode a peptide, polypeptide or protein.

A “deletion” is an event in which regions of DNA sequence present in the original plasmid copy of the viral vector genome are lost during the process of reverse transcription. As such the deleted sequence is absent from some or all of the single stranded RNA molecules transcribed from the original plasmid during the packaging process in which particles of replication incompetent lentiviral vectors are produced. Note, the plasmid DNA sequence remains intact at all times, deletion occurs during the process of transcription during the process of packaging whereby two copies of single strand RNA are reverse transcribed and assembled within a protein coat.

Furthermore, an unmodified nucleic acid sequence or polypeptide that is not normally expressed in a cell is considered heterologous. Vectors of the invention can have one or more exogenous DNA sequences inserted at the same or different insertion sites, where each is operably linked to a regulatory nucleic acid sequence which allows expression of the sequence. Thus, vectors resulting from the invention may be used to express various types of proteins, including, e.g., monomeric, dimeric and multimeric proteins.

The vectors described in the present invention can be used to express a “heterologous protein”.

As used herein, the term “heterologous” means a nucleic acid sequence or polypeptide that originates from a foreign species, or that is substantially modified from its original form if from the same species.

A suitable heterologous peptide may be a recombinant protein which has therapeutic activity or other commercially relevant applications. Examples of heterologous proteins which may be expressed include; cytokines such as interferon alpha, beta and/or gamma, interleukins, and hematopoietic factors such as Factor VIII. In one embodiment, the heterologous peptide may encode for an antibody heavy chain or light chain, which can be of any antibody type, e.g. murine, chimeric, humanized and human, where the two chains can come from the same or different antibodies.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

### BRIEF DESCRIPTION OF THE DRAWINGS AND DETAILED DESCRIPTION

The present invention will now be described with reference to the following examples which are provided for the purpose of illustration and are not intended to be construed as being limiting on the present invention. Reference will further be made to the accompanying drawings in which:

FIG. 1 shows the full DNA sequence of the R24 minibody used in the construction of pRI28 and pLE38. The start codon and double stop codons are capitalised,

FIG. 2 shows the schematic structure of R24 minibody,

FIG. 3, plasmid map of the lentiviral vector genome, pRI28,

FIG. 4 shows the complete DNA sequence of the lentiviral vector genome plasmid, pRI28,

FIG. 5 shows the predicted structure of the RNA genome of the pRI28 virus,

FIG. 6 shows a diagram with the relative positions of some of the deletions (subsequently referred to by unique ‘lt’ numbers) identified within the R24 coding sequence in the lentiviral vector pRI28,

FIG. 7 shows a schematic representation of the predicted structure of the RNA genome of pLE38,

FIG. 8 shows the full sequence of the 3′ end of the pLE38 genome encompassing the complete R24 coding sequence (shown in bold text with start and double stop codon capitalised). The 5′ LTR sequence is also shown in bold text. Both copies of the lt1 repeat are italicised and the sequence lost after the lt1 deletion event is underlined. Note the 5′ copy of the lt1 repeat is retained after deletion and as such is not underlined,

FIG. 9 shows the R24 minibody V_{H} domain amino acid sequence. The amino acid sequence of R24 minibody is shown in single letter code. Italicised letters indicate those residues at 5′ and 3′ ends of this region that lie outwith the FR and CDR designations. Bold text shows the residues comprising the three framework regions (key in box to the right of figure). Standard text shows the residues comprising the CDRs. Underlined text shows the amino acid residues that are coded for by problematic DNA repeats,

FIG. 10 shows the R24 minibody V_{L} domain amino acid sequence. The amino acid sequence of R24 minibody is shown in single letter code. Italicised letters indicate those residues at 5′ and 3′ ends of this region that lie outwith the FR and CDR designations. The residues of the linker domain are italicised at the 5′ end. Bold text shows the residues comprising the three framework regions (key in box to the right of figure). Standard text shows the residues comprising the CDRs. Underlined text shows the amino acid residues that are coded for by problematic DNA repeats,

FIG. 11 shows the eight potentially problematic sequences in the R24 minibody and associated deletions (referred to by individual lt numbers),

FIG. 12 shows a diagram of the 3′ end of the genome in pLE38. *indicates the position of two short repeat sequences referred to as “lt1” that are implicated in some of the deletions occurring within the R24 coding sequence. The position of two BspEI sites flanking the 5′ lt1 repeat, the replacement sequence in which the lt1 sequence has been removed, is indicated by a thick black line,

FIG. 13 shows the full sequence of the BspEI fragment inserted into pLE38 during the lt1 repair process, restriction sites shown in bold text,

FIG. 14 contains a table showing a comparison between the eight problematic regions in the R24 minibody and the equivalent residues in the anti-CD55 minibody,

FIG. 15 shows the DNA and amino acid sequence encoded by both the original and the modified linker present in standard R24 and the repaired version,

FIG. 16 shows the primary amino acid sequence of the optimised anti-CD55 minibody,

FIG. 17 shows the DNA sequence of the optimised anti-CD55 minibody,

FIG. 18 shows a comparative diagram of the relative structures of an antibody versus a minibody,

FIG. 19 shows the primary amino acid sequence of the heavy chain of the anti-CD55 antibody,

FIG. 20 shows the primary amino acid sequence of the light chain of the anti-CD55 antibody,

FIG. 21 shows a plasmid map of pLE121, the anti-CD55 antibody heavy chain as supplied by Geneart in the pCRscript vector,

FIG. 22 shows a plasmid map of pLE120, the anti-CD55 antibody light chain as supplied by Geneart in the pCRscript vector,

FIG. 23 shows the full sequence of the 3′ end of the pLE119 genome encompassing the complete anti-CD55 coding sequence (shown in bold text with start and double stop codon capitalised). The 5′ LTR sequence is also shown in bold text. Both copies of the lt230 repeat are italicised and the sequence lost after the lt230 deletion event is underlined. Note the 5′ copy of the lt230 repeat is retained after deletion and as such is not underlined,

FIG. 24 shows a revised version of the table given in FIG. 11 in which the problematic repeat sequences determined from work with both R24 and anti-CD55 are listed,

FIG. 25 shows an ethidium bromide stained 1% agarose gel of PCR products amplified from genomic DNA of cells individually transduced with pLE118 and pLE119. PCR primers amplify the 3′ end of each genome, from within the candidate tissue promoter to the 3′ LTR encompassing the entire heavy or light chain coding sequences. The 2124 bp and 1398 bp products amplified from pLE118 and pLE119 transduced cells respectively are diagnostic of the presence of the intact anti-CD55 coding sequences. Note the absence of smaller amplification products,

FIG. 26 shows two tables summarising the codon usage frequencies in chicken (Gallus gallus) and quail (Coturnix coturnix).

### EXAMPLE 1

### The R24 Minibody —RT-PCR Data

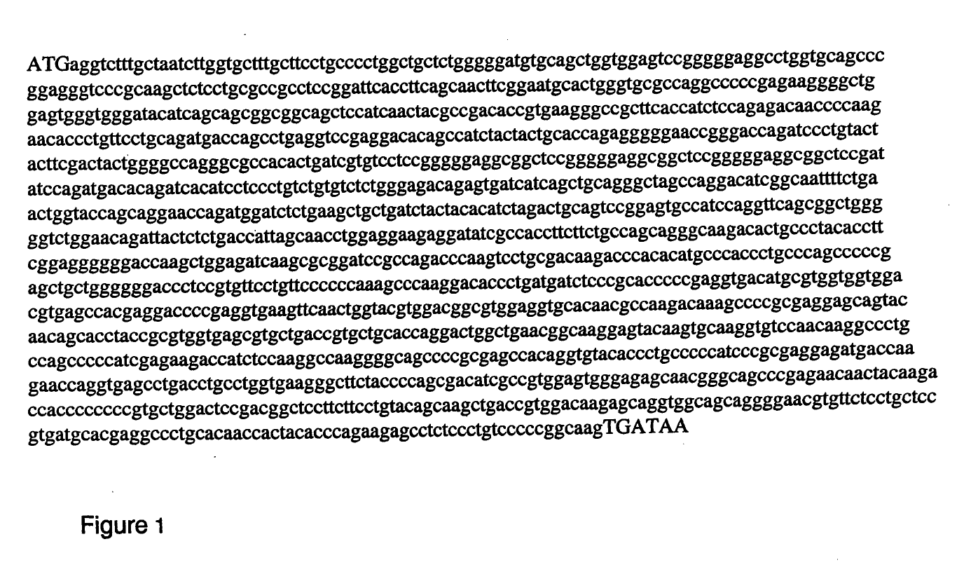

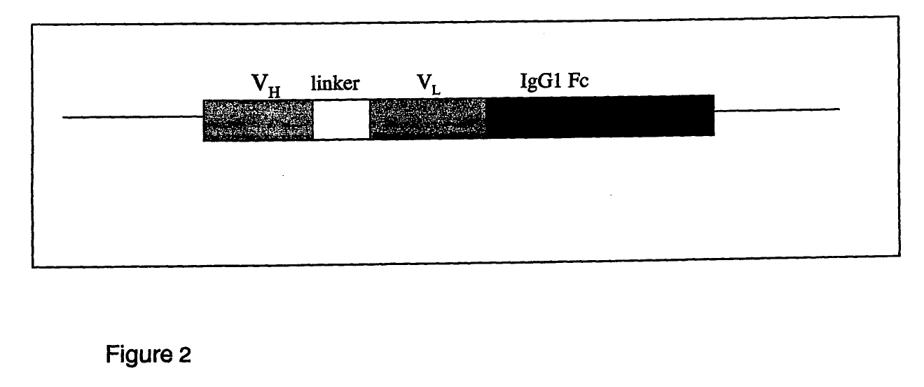

The full sequence of the R24 minibody used with the EIAV lentiviral vector is shown in FIG. 1. This recombinant antibody molecule consists of a standard scFV fragment, comprised of a mouse V_{H}, a linker and a mouse V_{L}, inserted upstream of the human IgG1 Fc domain (FIG. 2). This sequence was introduced downstream of two types of promoter, first a global promoter; the human Cytomegalovirus virus (hCMV) immediate early promoter. Second, a candidate tissue-specific promoter designed to actively express the R24 minibody in a spatio-temporally restricted manner within a transgenic avian.

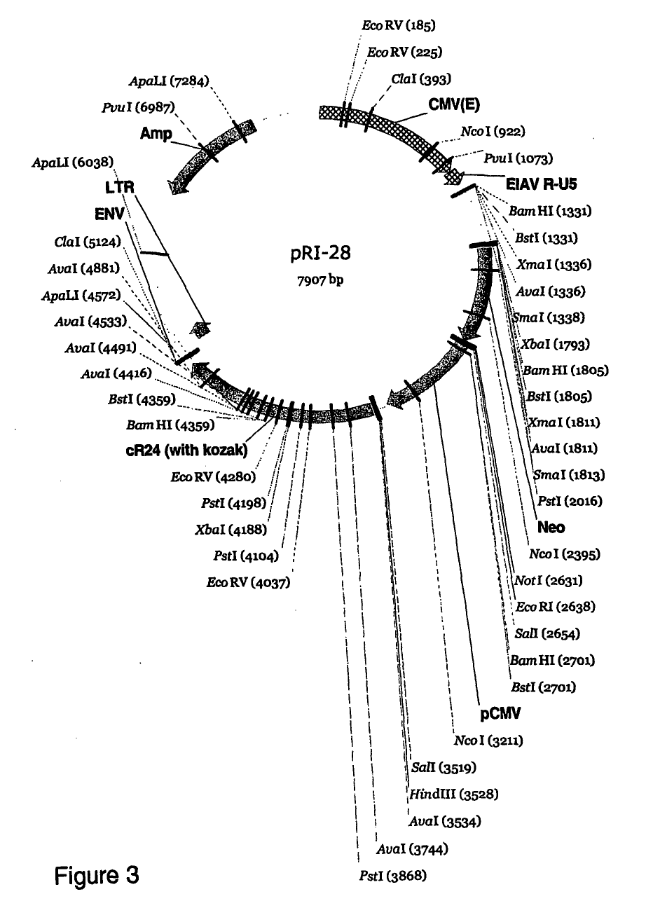

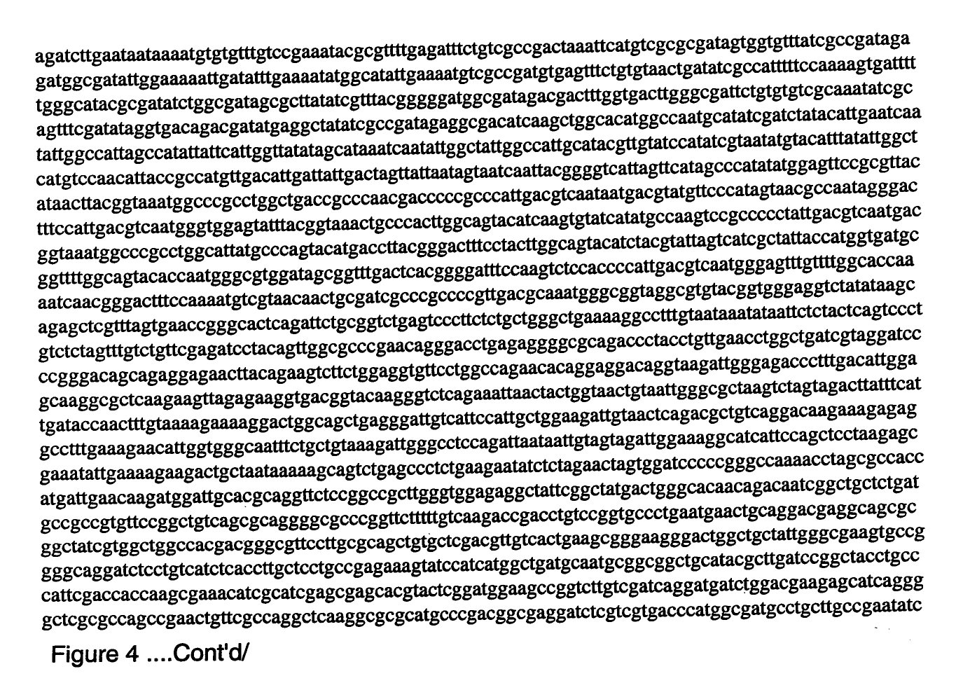

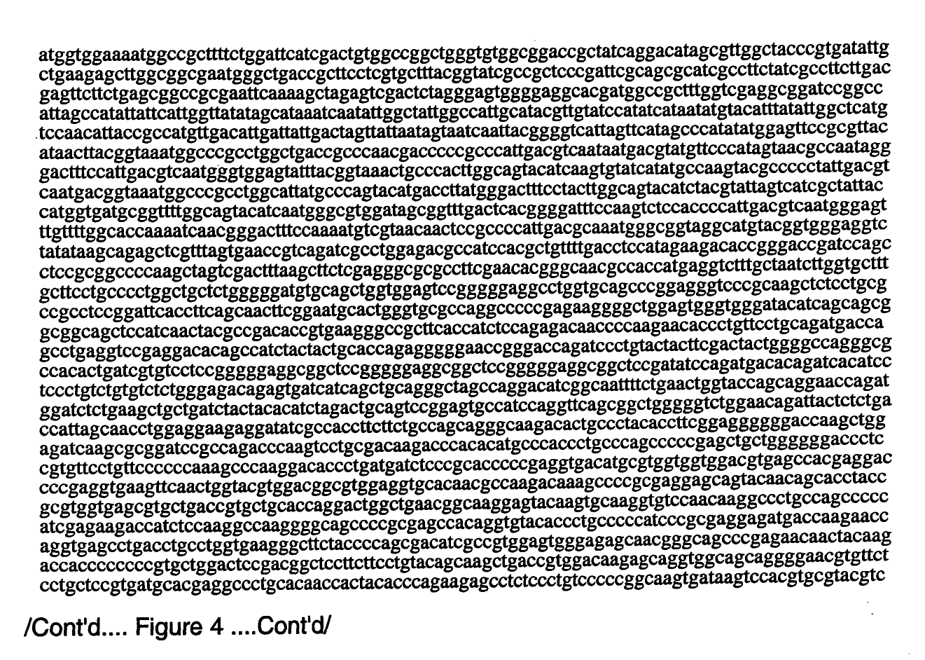

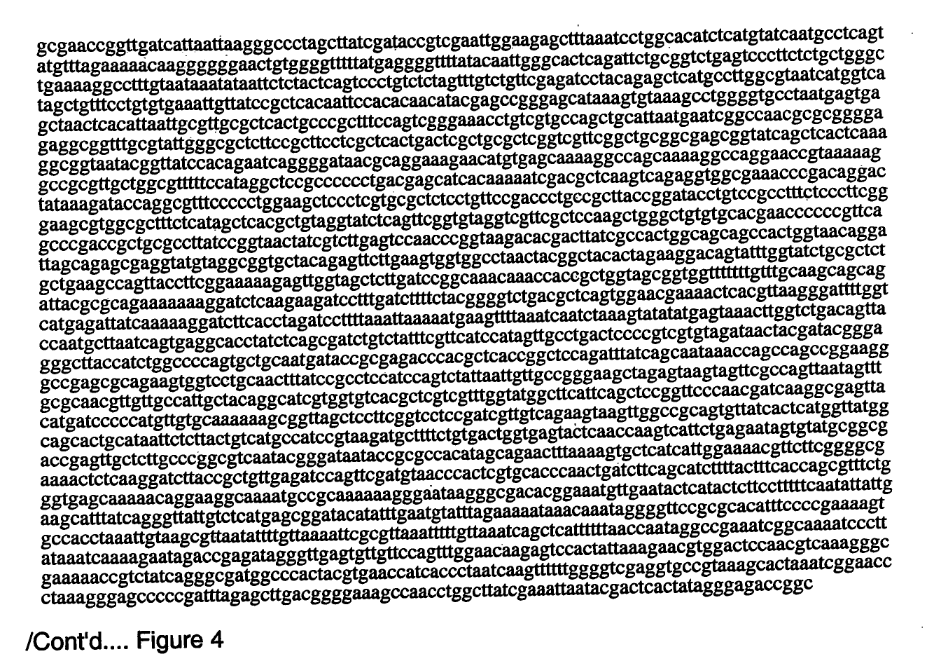

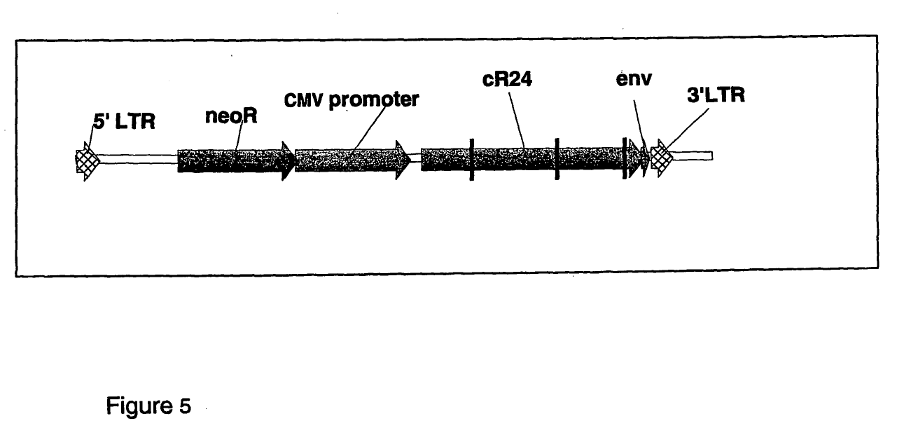

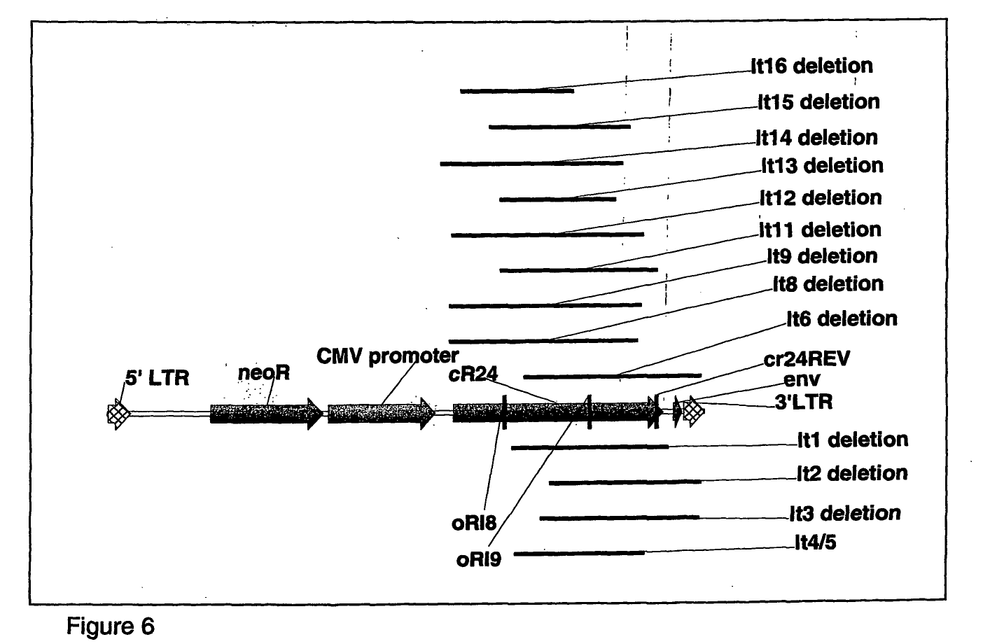

R24 was inserted downstream of the hCMV promoter to generate the viral genome plasmid pRI28 (Plasmid map given in FIG. 3, full sequence given in FIG. 4). Transient transfection of this genome plasmid into D17 canine osteosarcoma cells and subsequent ELISA on the cell medium demonstrated a secreted human IgG1 level of 600 ng/ml. This result confirmed the expression-competence of the pRI28 genome. Packaged replication incompetent RNA genomes of pRI28 were obtained via standard transfection techniques. D17 cells were then transduced with pRI28 virus. Medium harvested from these cells was then analysed by ELISA and no secreted human IgG1 was detected. Viral RNA was also harvested from the packaged virus and the structure of the pRI28 genomes was analysed by RT-PCR. RT-PCR demonstrated that a mixed population of genomes were present in a sample of packaged pRI28 virus, all of which were transcribed from a homogenous preparation of pRI28 plasmid. The most significant differences were found at the 3′ end of the genome (FIG. 5) from where apparently full-length and truncated products could be amplified. Numerous apparently truncated RT-PCR products were cloned and sequenced and deletion events were confirmed as encompassing some or all of the R24 coding sequence. The position of some of these deletion events is shown in FIG. 6 (subsequently referred to by unique ‘lt’ numbers). Note, given the nature of the deletion events shown in FIG. 6 such genomes would be predicted to be unable to express the R24 minibody.

Careful analysis of these lt deletion events demonstrated that the deletions were delineated by small (5-10 bp) direct repeats. The results identify these sequence elements as being potentially non-EIAV compatible.

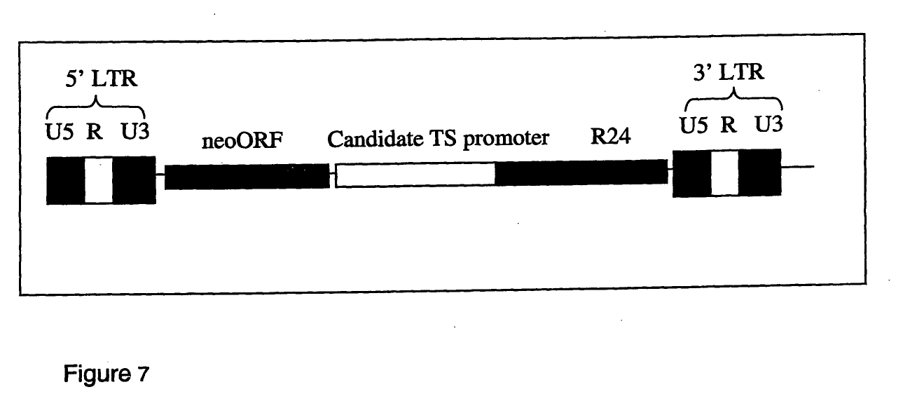

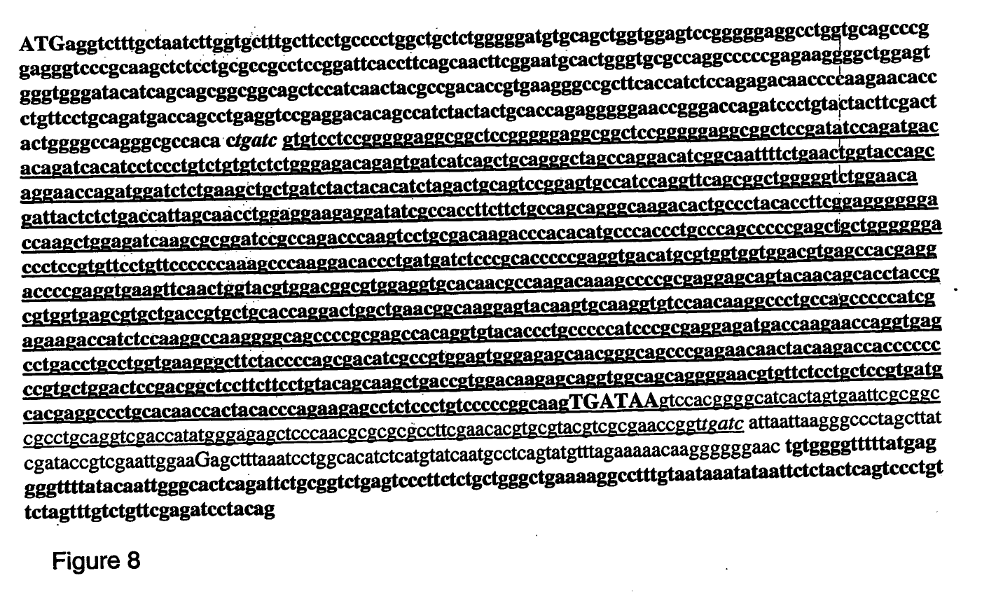

The role of short, direct repeat elements in transgene deletion events was further confirmed by work on a related viral genome. The same R24 minibody coding sequence was inserted downstream of a candidate tissue-specific promoter to generate the plasmid pLE38 (schematic genome map given in FIG. 7). Packaged replication incompetent RNA genomes of PLE38 were obtained via standard transfection techniques. RT-PCR analysis was completed exactly as described for pRI28 and as with pRI28, apparently truncated PCR products were amplified from the 3′ end of the viral genome encompassing some or all of the R24 coding sequence. Cloning and sequence analysis of the PCR products indicated a prevalence of one particular deletion product, lt1, also previously detected in pRI28 virus (see FIG. 6, deletion map). The full sequence of the lt1 deletion product is given in FIG. 8.

### EXAMPLE 2

### Interpretation of the R24 Minibody Sequence Data from pRI28

In the R24 minibody, there are two categories of such potentially problematic short, direct repeat sequences, those within the scFV region itself (V_{H}, linker and V_{L}) and those within the IgG1 Fc domain. The schematic structure of the R24 minibody is shown in FIG. 2.

### V_{H} Domain

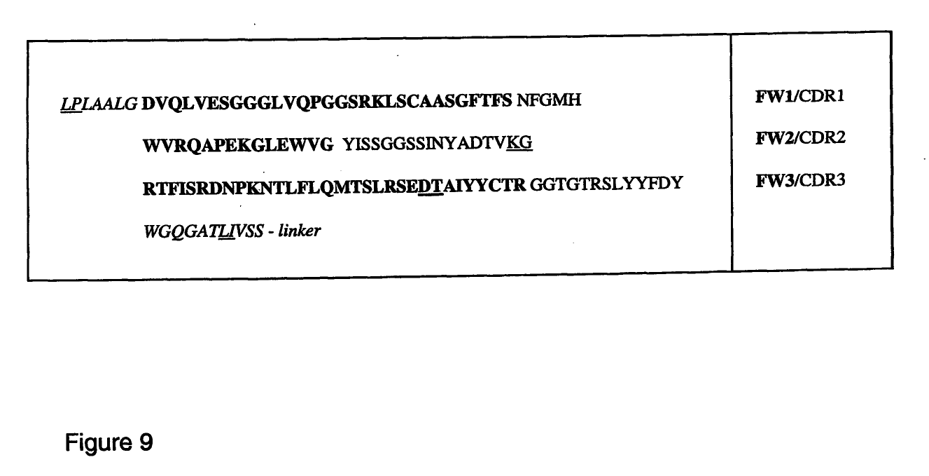

Four problematic repeats were identified in the R24 minibody sequence within V_{H}—the first lies at the extreme 5′ end (LP, Leu Pro in FIG. 9, involved in deletion lt16), the second lies within CDR2 (KG, involved in deletion lt15), the third in FW3 (DT involved in deletion lt11 and 13) and the fourth at the 3′ end of V_{H} prior to the linker sequence (LI, involved in deletion lt1).

### Linker/V_{L} Domain

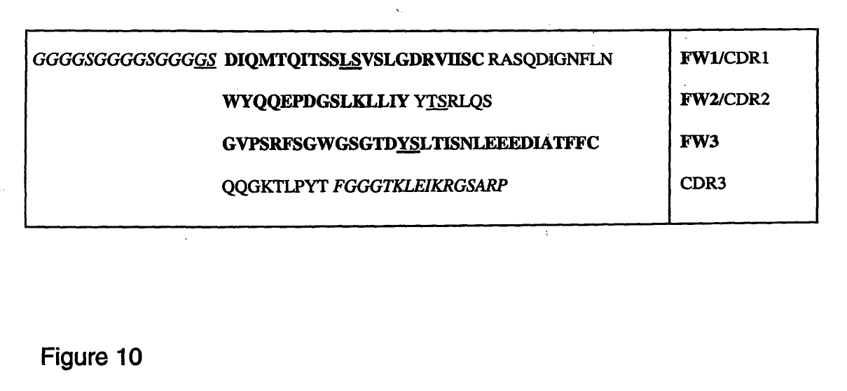

Four problematic repeats were identified in the linker and V_{L} domain. The first lies within the linker (GS in FIG. 10, involved in deletion lt4 and 5), the second lies within FW1 (LS, involved in deletion lt6), the third in CDR2 (TS involved in deletion lt3), and the fourth in FW3 sequence (YS, involved in deletion lt2).

### IgG1 Fc

The above sections have covered deletions that spanned from R24 minibody to 3′ virally-derived sequences. Sequences underlined represent the 5′ end of those deletions. However, deletions possibly arising due to recombination events between the R24 minibody and sequences to the 5′ of the gene were also detected. In these instances the 3′ determinants were located within the IgG1 Fc domain of R24 minibody. Two proline-rich tracts have now been identified within this sequence as being involved with or adjacent to these deletions.

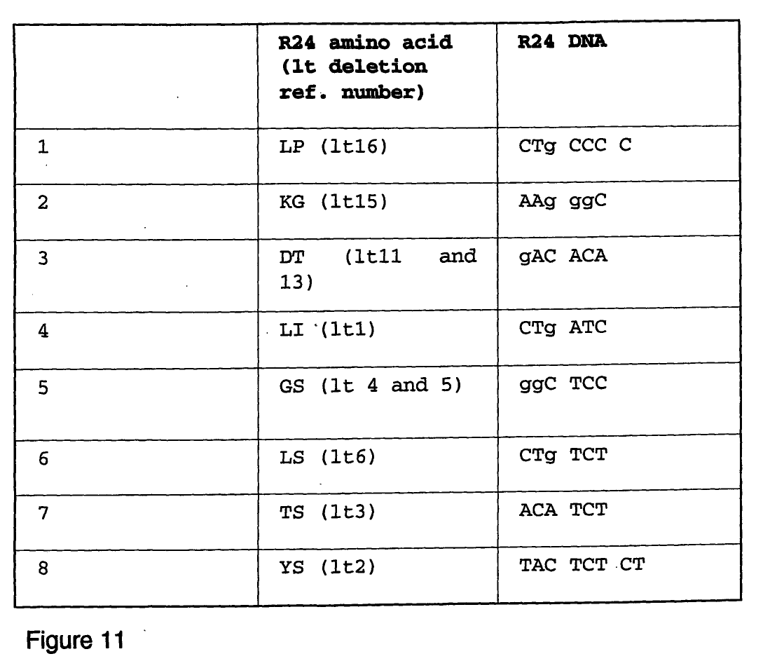

The eight potentially problematic sequences in the R24 minibody and associated deletions (referred to by individual it numbers) are summarised in FIG. 11. It is the short, direct repeat sequences that delineate these deletions that are removed from candidate transgenes during the analysis previously described in step (iii). Using Vector Nti software (Informax Inc., Invitrogen) or equivalent, DNA sequences can be screened for the presence of these sequences. If the transgene is not a recombinant antibody then it is unlikely that all of these residues will be conserved. The transgenic avian expression system may be able to express recombinant antibodies, in which case these residues may be conserved, particularly as some occur within framework regions (FR)— variable domain sub-regions known to show more conservation than those residues in complementarity determining regions (CDRs).

This is also relevant to the IgG1 Fc that is the effector domain of choice for many commercial recombinant antibodies and so will be absolutely conserved in many candidate transgenes. Work with the R24 minibody has shown that several deletion determinants may be located within this domain, for example, two proline-rich protein regions encoded by poly-pyrimidine tracts of DNA are consistently involved with or adjacent to these deletions. Therefore, it is recommended that these poly-pyrimidine tracts be removed. Since the chicken uses four codons to encode Pro/P with almost equal frequency it is possible to alternate codon usage to remove poly-pyrimidine tracts in the DNA sequence while still encoding for multiple proline residues in the resultant protein.

### EXAMPLE 3

### “Repaired” R24 Minibody

To try and establish the relevance of short, direct repeats and associated deletions it was decided to remove the lt1 sequence (5′CTG ATC 3′) from the R24 minibody sequence and simultaneously replace the linker with the non-repetitive sequence. The effects of this repair were then tested in the vector designated as pLE38 as the lt1 deletion event had been shown to be present in a significant proportion of packaged RNA genomes.

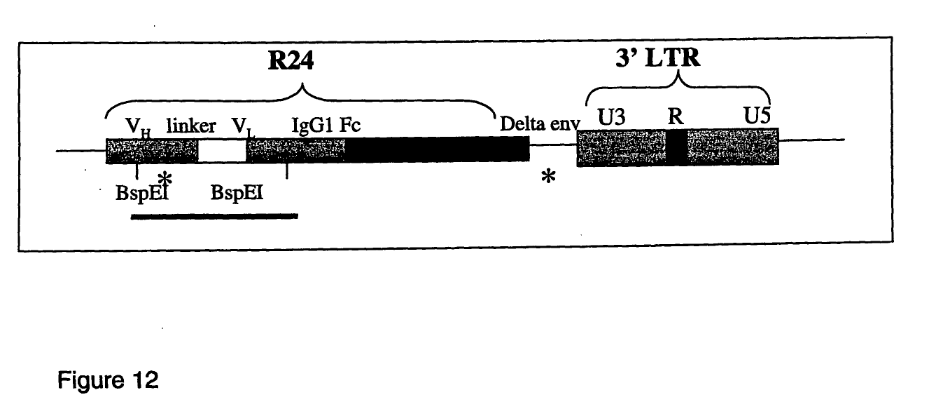

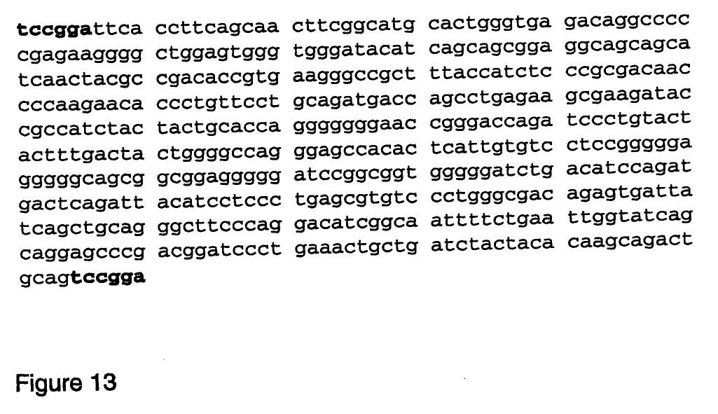

Digestion of pLE38 with the restriction enzyme BspEI allows a removal of the 5′ lt1 repeat sequence and old linker, and replacement with a new piece of DNA encoding the new linker and in which the lt1 sequence has been removed (see FIG. 12). The full sequence of the replacement segment of DNA inserted into pLE38 to generate “repaired R24” is given in FIG. 13. The completed plasmid was called pLE56.

The set of two plasmids, repaired and unrepaired were then packaged side by side and the structure of RNA genomes and integrated transgenes in the genomic DNA of transduced cells was analysed by PCR.

### EXPERIMENTAL DATA

### pLE38 and pLE56

Real time qPCR analysis of the viral RNA from the repaired R24 minibody demonstrated that an apparently acceptable level of this genome had been successfully packaged and that the lt1 repair did not have a detrimental effect on titre. ELISA analysis failed to detect R24 minibody expression but this is a positive result as, in theory, expression from the promoter contained in this vector should be tissue-specific and we would not expect the promoter to be active in vitro. Real time qPCR conducted on genomic DNA from cells transduced with these viruses successfully amplified a product spanning the EIAV packaging signal thereby confirming the transduction status of the cells providing more evidence that a lack of leaky ovalbumin promoter activity rather than a lack of integration explains the negative ELISA result.

Furthermore, a PCR reaction spanning the 3′ end of the genome in both viruses successfully amplified a full-length product from the genomic DNA of cells transduced only with pLE56. This is in direct contrast to the predominant amplification of the lt1 deletion product from the packaged RNA genome of pLE38 (unrepaired). However, the lt1 repair alone was insufficient in the pLE38 test system to abolish the presence of smaller, putative deletion products. The most probable explanation for this result is the presence of other potentially problematic short, direct repeat elements still retained within the “repaired” R24 as only the 5′ lt1 repeat had been removed. This possibility can only be explored by first, an evaluation of whether the potentially non-EIAV compatible sequences listed in FIG. 11 are applicable to other transgenes and second; an evaluation of internal deletion frequencies in a transgene in which all potentially non-EIAV compatible sequences have been removed.

### Instability in Bacteria

Anecdotal evidence has indicated that the previous linker sequence used in R24 minibody was unstable in bacteria. Deletions of individual repeat elements were detected. No such problems have been encountered with the new linker that has been successfully cloned into numerous expression vectors, such as pLE56.

### EXAMPLE 4

### Anti-CD55 Minibody (791T/36)

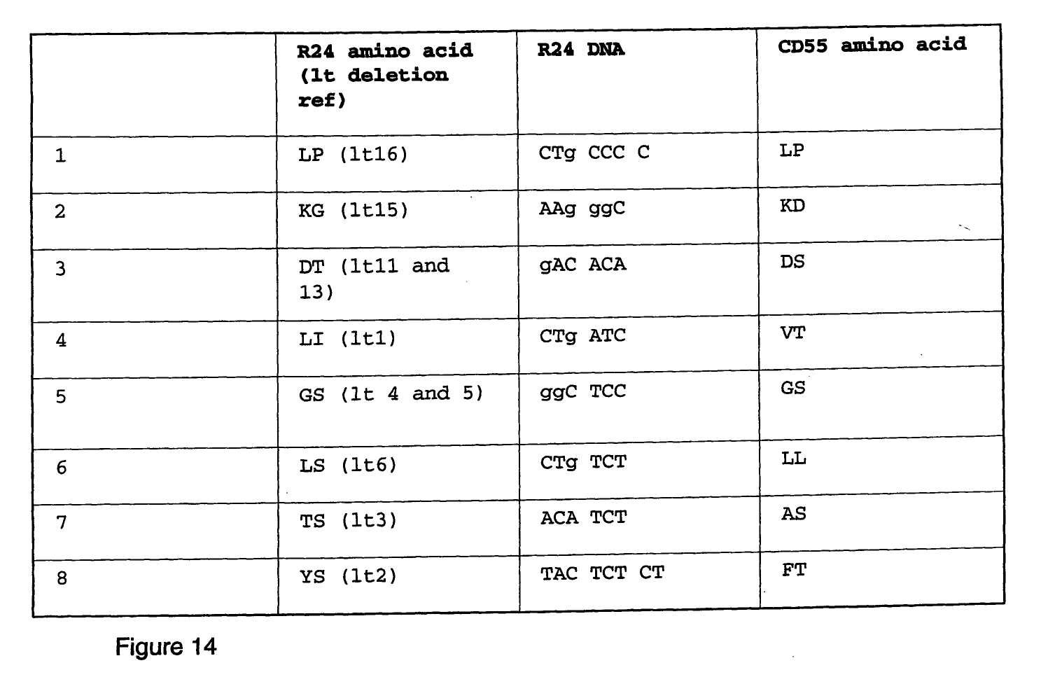

Numerous potentially non-EIAV compatible sequences have been identified as a consequence of work with the R24 minibody. It was of interest to determine whether such sequences would be present in a non-R24 based transgene. Therefore, the anti-CD55 minibody DNA sequence was assessed in order to determine whether the potentially non-EIAV compatible sequences identified in R24 could be applied to another transgene and as such if deletions would be predicted to occur in its sequence when incorporated into an EIAV lentiviral vector backbone. A direct sequence comparison was carried out between this minibody and the R24 minibody. Eight problematic regions were identified in the minibody and these regions are summarised in FIG. 14.

Line 1 of the table of FIG. 14 shows a perfect match between the residues involved in the lt16 deletion event in the R24 minibody and the CD55 minibody. This is because these residues are encoded by the basic lysozyme signal peptide shared by both constructs. Codon usage of the signal peptide has been modified prior to the synthesis of another transgene, a cytokine-based product. Although the lt16 repeat is still present in the modified signal peptide no equivalent lt16 deletions have been identified in another gene construct based on the interferon beta gene, thus far analysed. Therefore, it would appear that the presence of the lt16 repeat alone, at least in non-minibody containing vectors, is insufficient to cause deletion and another factor must be involved, for example the linker domain. However, it is advisable that codon usage is further modified in the signal peptide to remove this element.

Line 2 of the table of FIG. 14 shows that only one of two amino acids match between R24 minibody and CD55 minibody (KG versus KD). The chicken uses two codons for Lys/K with almost equal frequency so it would be possible to change the codon but retain the amino acid specificity and remove the lt15 repeat element from anti-CD55.

Line 3 of the table in FIG. 149 shows that only one of two amino acids match between the R24 minibody and CD55 minibody (DT versus DS). As with Lys/K above, the chicken uses two codons for Asp/D with almost equal frequency, so again it would be possible to change the codon but retain the amino acid specificity and remove the lt11/13 repeat element from anti-CD55 minibody.

Line 4 of this table refers to the LI sequence that encodes the most problematic lt1 repeat in the R24 minibody. This deletion has now been identified in two R24-minibody-based lentivectors, pRI28 and pLE38. Fortunately, there is no sequence homology at this point with anti-CD55 minibody.

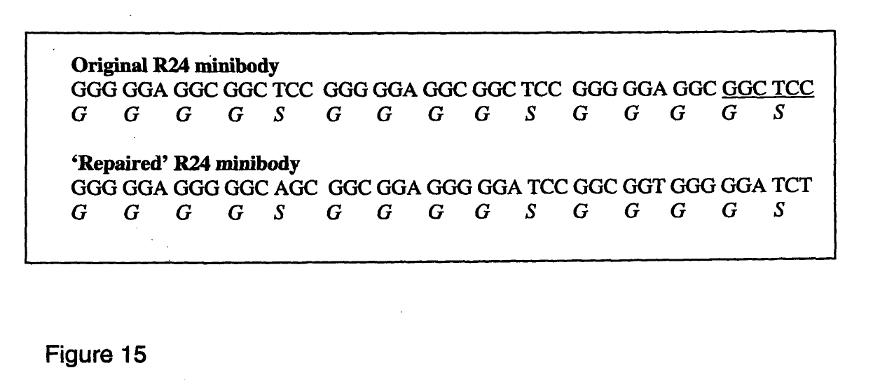

Line 5 of this table shows a perfect match between the residues involved in the lt4 and 5 deletion events in the R24 minibody and anti-CD55 minibody. This is because the linker used to join the V_{H} and V_{L} domains during the construction of the scFV component of the minibody encodes these residues. Several lines of evidence indicate that this linker may be sub-optimal for use in expression studies; anecdotal evidence indicating repeat instability in E. coli, possibility of secondary structure given the three direct repeats in the linker, discussions with Geneart and literature on repeats and RNA polymerase interaction. The linker in the R24 minibody can be replaced with a new linker as shown in FIG. 15. This retains the (GGGS)₄ amino acid pattern but alters codon usage to minimize homology.

Underlined text highlights the problematic sequence in the original linker; GGC TCC is actually repeated three times. In the new linker the direct repeats are abolished, the GGC TCC sequence never occurs and its replacement GGA TCT occurs only once. It is recommended that this new linker be used during gene synthesis of the anti-CD55 or any other scFV or minibody for use in the EIAV lentivector system.

Line 6 of FIG. 14 shows that there is a one in two match between R24 minibody and anti-CD55 minibody for the lt6 repeat (LS versus LL). The chicken favours the CTG codon for Leu so it may be best not to alter this sequence. Line 7 also shows that there is a one out of two match between R24 and anti-CD55 for the lt3 repeat (TS versus AS). The chicken uses six different codons for Ser/S so there are several alternatives that can be used effectively to remove the lt3 repeat element. Finally, line 8 shows that residues YS involved in the lt3 deletion in R24 minibody are not conserved in anti-CD55 minibody so no sequence modifications would be required at this position (YS versus FT).

### IgG1 Fc Domain

It is also recommended to remove two multi-proline tracts within this Fc domain. Because the chicken uses four codons to encode Pro/P with almost equal frequency it will be possible to alternate codon usage to remove poly-pyrimidine tracts in the DNA sequence while still encoding for proline residues in the resultant protein.

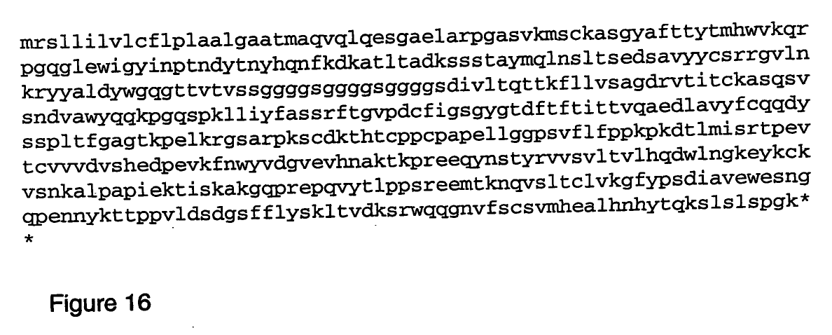

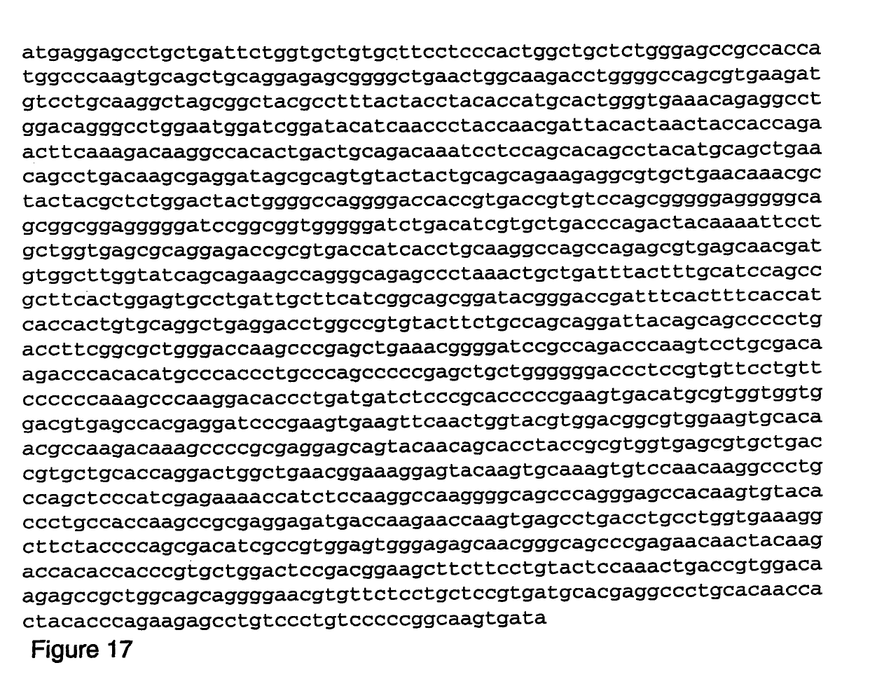

All of the above recommendations have been used to generate the optimal anti-CD55 minibody sequence for use in an EIAV lentivector given our current state of knowledge. Such optimised sequences are shown in FIGS. 16 and 17.

It is notable that the primary amino acid sequence is unchanged from that originally isolated, although the DNA sequence has been significantly altered. New 5′ and 3′ extensions have been added to facilitate gene expression in the avian transgenic test system, and a new linker has been introduced to abolish the direct repeats present in the equivalent R24 minibody molecule. All repeat motifs identified as potentially problematic have been removed, both at conserved positions between the R24 minibody and the anti-CD55 minibody and all other places within the coding sequence.

In conclusion, this analysis of the anti-CD55 minibody coding sequence has indeed demonstrated the relevance of this transgene optimisation methodology to non-R24 based transgenes.

### EXAMPLE 5

### Anti-CD55 Antibody (791T/36)

The data presented in Example 4 of this document demonstrated that the principle of removing potentially non-EIAV compatible short, direct repeat sequences is applicable to a non-R24 based molecule, in this case an anti-CD55 minibody. The next phase of this work was to evaluate the frequency of internal deletions within a transgene sequence present in an EIAV lentiviral vector after the processes of sequence optimisation have been applied exactly as described herein.

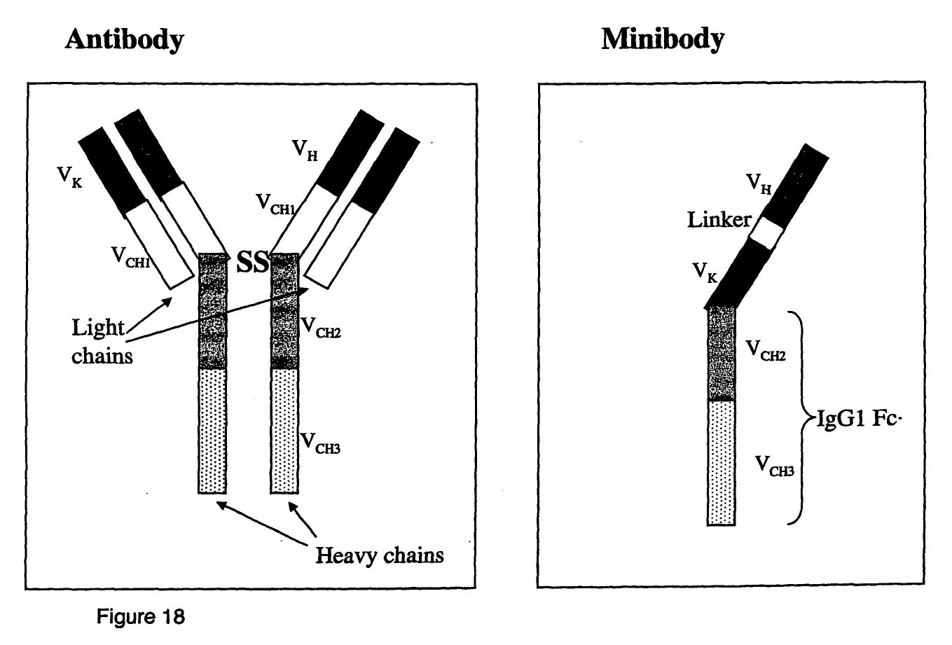

However, rather than generate transgenes encoding the anti-CD55 minibody described in Example 4, it was decided to apply the same principles of transgene optimisation to a double chain mouse/human chimaeric, anti-CD55 antibody. FIG. 18 contains a diagrammatic representation of the structures of both of these molecules.

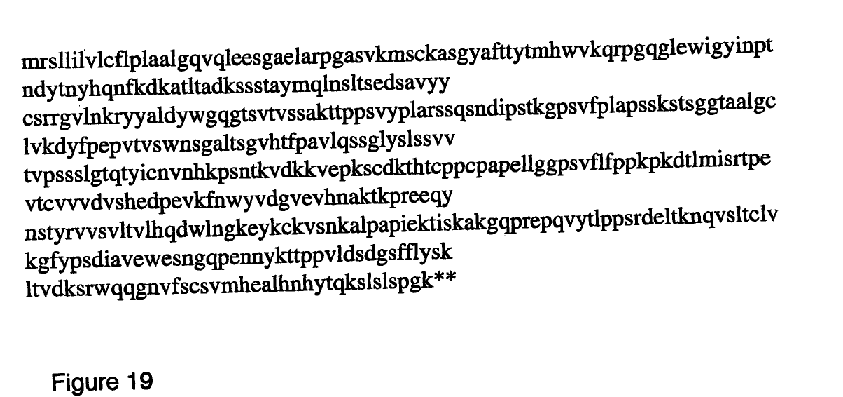

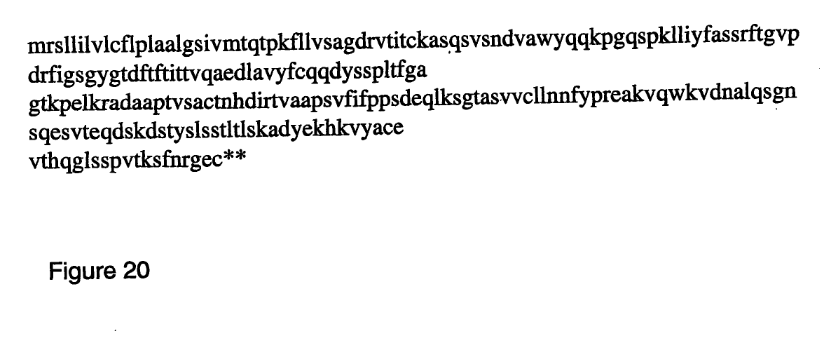

The chimaeric antibody consists of the mouse variable regions from both the heavy and light chain inserted upstream of the human IgG1 heavy chain and the human kappa light chain respectively. The primary sequences of both molecules were assembled in silico prior to the staged process of transgene optimisation described herein. FIGS. 19 and 20 show the primary amino acid sequence of the chimaeric heavy and light chains respectively. Note, both primary amino acid sequences contain a 5′ extension to add the signal peptide from the endogenous chicken lysozyme gene in order to allow secretion of both proteins.

The process of optimisation was carried out in accordance with the steps defined in the first aspect of the invention, namely; Geneart (Germany) was supplied with the desired primary amino acid sequences and DNA codons were assigned based on chicken codon usage preferences, a process referred to as ‘chickenisation’. Step (ii) of the optimisation process was then completed whereby the basic chickenised sequence was analysed to detect any elements predicted to have a negative effect on gene expression such as negative elements or repeat sequences, cis-acting motifs such as splice sites, internal TATA boxes or ribosomal entry sites. All such elements were removed via sequence modification. This second generation chickenised sequence was then analysed to identify and remove all potentially problematic sequences as those shown in FIG. 11 (Step (iii) of the optimisation process). The third generation sequence was sent back to Geneart to confirm these modifications had not re-introduced any elements predicted to have a negative effect on gene expression such as negative elements or repeat sequences, cis-acting motifs such as splice sites, internal TATA-boxes or ribosomal entry sites. This process was iterative with all changes designed to remove potentially problematic repeat sequences checked to ensure codon usage was still optimal and that no negative elements had been re-introduced. A final version of the chimaeric anti-CD55 heavy chain and light chain was then generated via gene synthesis.

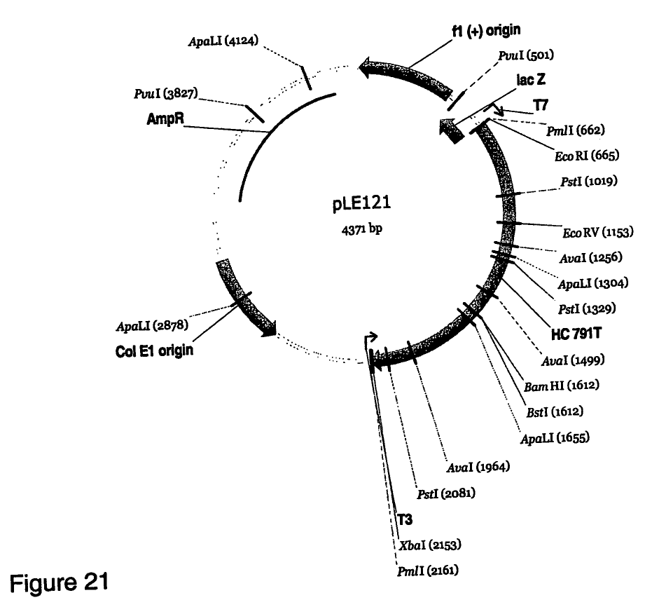

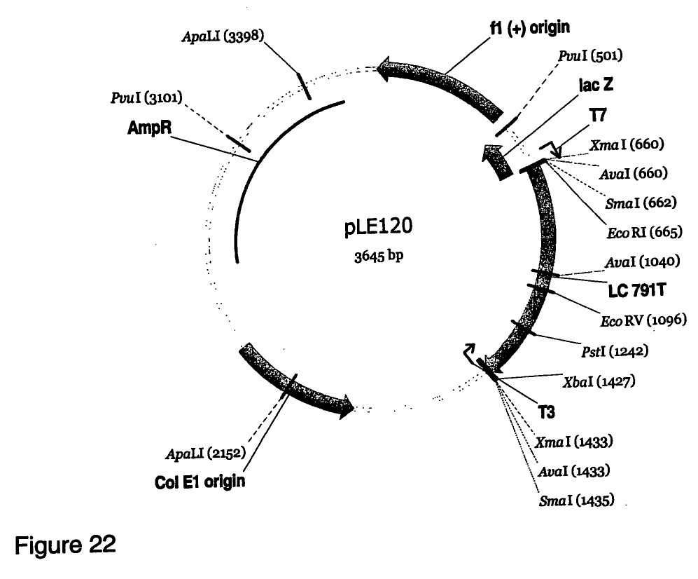

Both anti-CD55 coding sequences were supplied in individual pCRScript vector backbones and could be excised via digestion with the restriction enzymes PmlI, heavy chain (FIG. 21, pLE121), and SmaI, light chain (FIG. 22, pLE120). The ability of an EIAV lentiviral vector system to support the expression of the optimised transgenes was then analysed by constructing vector genomes in which the transgenes were introduced downstream of a candidate tissue-specific promoter.

### Anti-CD55 Antibody and Candidate Tissue Specific Promoter-Based Expression Constructs

The heavy and light chain sequences were, separately, inserted downstream of a candidate tissue-specific promoter to generate the plasmids pLE118 and pLE119 respectively. The genome organisation of both pLE118 and pLE119 is identical to the schematic shown for pLE38 in FIG. 7 except that the relevant heavy or light chain sequences replace R24.

Viral genome packaging was completed using standard transfection techniques. Genome RNA was harvested and analysed by RT-PCR, furthermore, the virus particles were used to transduce host cells from which genomic DNA was then harvested. A PCR analysis of genome structure was then completed.

RT-PCR and subsequent cloning and DNA sequencing of the products amplified from packaged viral genomes suggested the presence of intact anti-CD55 heavy chain and light chain sequences within the packaged genomes of pLE118 and pLE119 respectively.

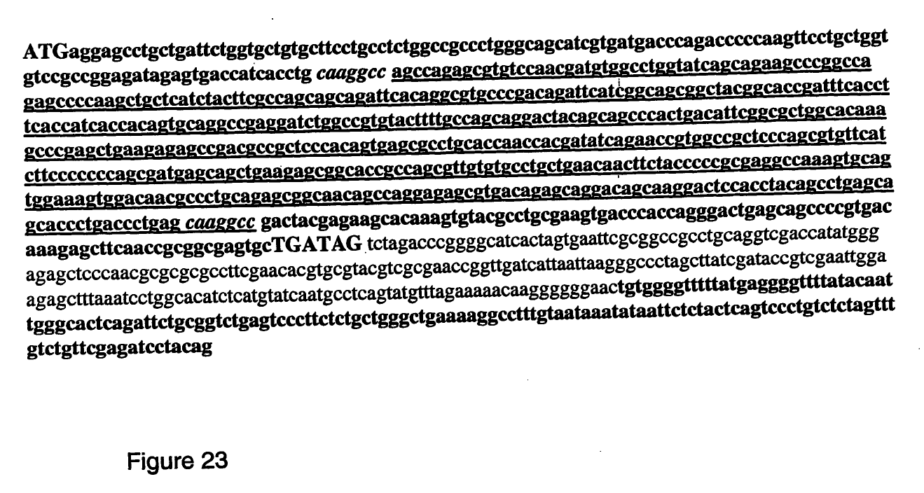

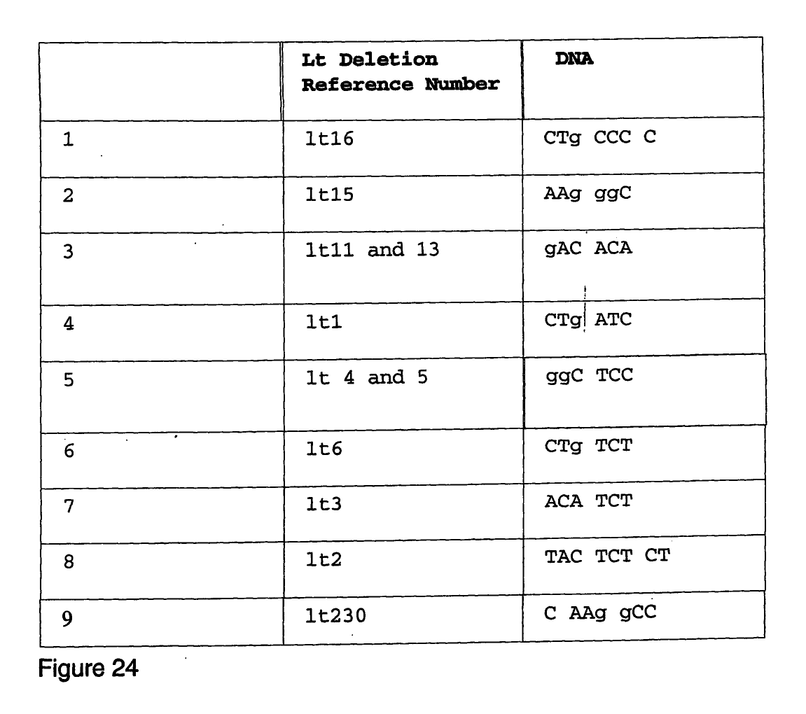

Interestingly one deletion product was identified from the pLE119 genome, referred to as lt230. The full sequence of the 3′ end of pLE119 is given in FIG. 23 with the extent of the lt230 deletion indicated. Note the presence of the short, direct repeats that delineate the 5′ and 3′ extent of this deletion. This data represents the first evidence for the occurrence of internal deletions within a non-R24 based EIAV lentiviral vector transgene by the putative homologous recombination-based mechanism outlined in this document. As such the lt230 flanking repeat sequence has now been added to the list of sequences that should be removed in step (iii) of the transgene optimisation process. All such sequences are listed in FIG. 24.

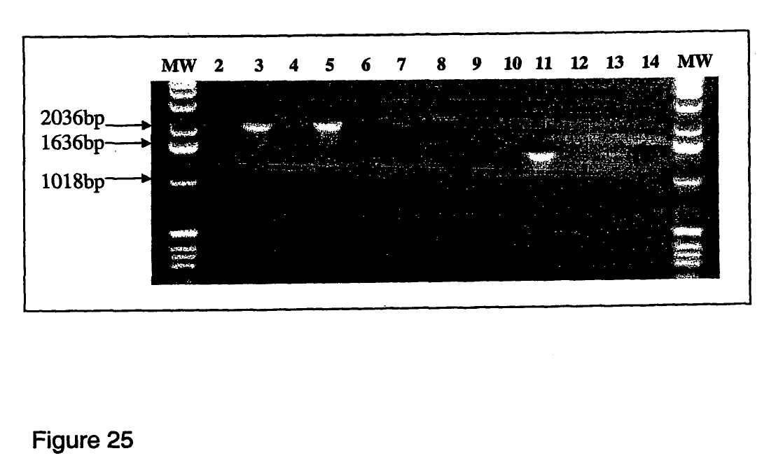

Analysis of the genomic DNA of pLE118 and pLE119 transduced cells yielded predominantly full-length amplification products. For example, a PCR reaction spanning from within the candidate tissue specific promoter to the 3′ LTR and encompassing the transgene coding sequence gave rise to a 2124 bp product diagnostic of the presence of intact heavy chain sequences, from the genomic DNA of cells transduced with pLE118 virus (lane 7, FIG. 25). The same PCR reaction gave rise to a 1398 bp product diagnostic of the presence of intact light chain sequences, from the genomic DNA of cells transduced with pLE119 virus (lane 13, FIG. 25). Note both transgene coding sequences share the same lysozyme-derived leader peptide hence the ability to use shared PCR primers. The lt230 deletion product was not amplified from the genomic DNA of cells transduced with pLE119 suggesting that it does not represent a majority species.

There are several conclusions to be drawn from this work. First, the successful PCR amplification of intact optimised antibody coding sequences from these vectors in contrast to the results obtained for R24. Second, the discovery of a novel lt deletion in the CD55 sequence. This application details a procedure to remove all potentially problematic sequences identified as a consequence of work with the R24 minibody. The failure to detect any of the deletion products seen with R24 in the anti-CD55 test system supports the conclusion that such sequences are directly involved in the deletion mechanism. For example, in an early iteration of the anti-CD55 light chain the lt16 repeat sequence (CTg CCC C) was present. This was identified during the screening process to remove these potentially problematic repeat sequences and in later iterations changed to CTg CCT C with the encoded amino acids remaining unchanged. Crucially no evidence of the lt16 deletion event was detected with the final optimised anti-CD55 light chain sequence in contrast to the R24 results described earlier.

However, the detection of a novel lt deletion in the anti-CD55 antibody sequence provides another potentially problematic sequence that will be removed in further transgenes optimised by the method disclosed herein.

### EXAMPLE 6

### Transferability to Other Species

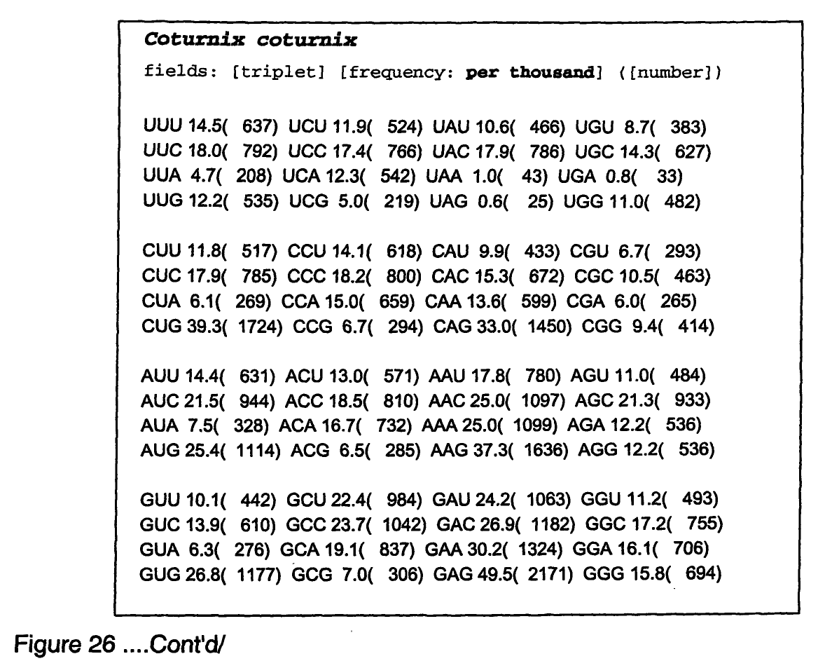

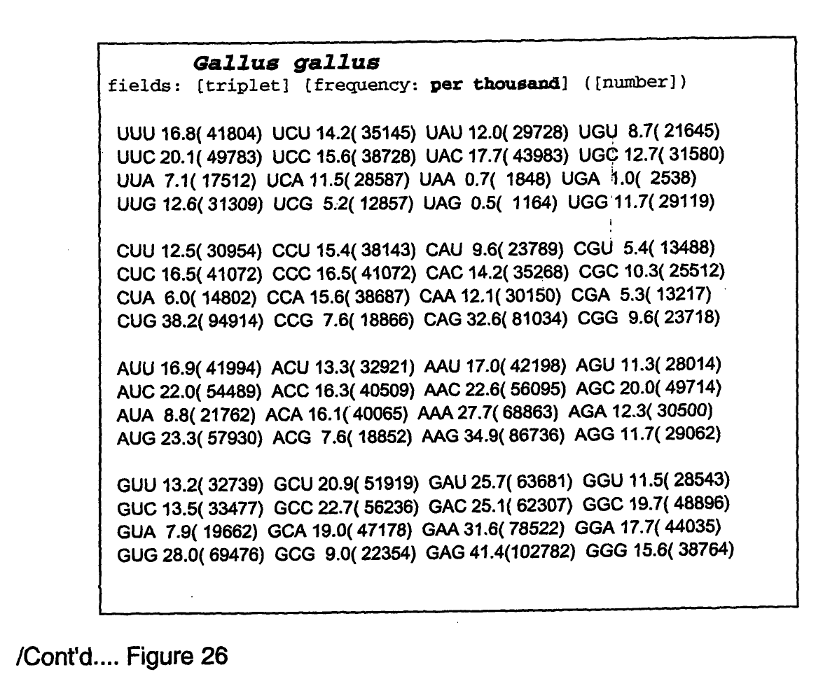

The process of transgene optimisation described here can be applied to heterologous coding sequences designed to be expressed in other species, for example, the Quail, Coturnix coturnix. As shown in FIG. 26 the codon usage frequencies in the Quail are almost identical to those in the chicken (Gallus gallus). As such the process of optimisation would be carried out in accordance with the steps defined in the first aspect of the invention. Namely, Geneart (Germany) supplied with the desired primary amino acid sequence and DNA codons assigned based on Quail or Chicken codon usage frequencies due to the very high degree of conservation in codon bias between these and other avian species. The optimisation process would then be completed whereby the basic sequence is analysed first, to detect any sequence elements predicted to have a negative effect on gene expression and second, to remove all potentially problematic sequences as shown in FIG. 24.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

Ch'ang LY, Yang W K, Myer F E, Koh C K, Boone L R (1989). Virology 168, 245-255.
Clements J E & Payne S L (1994) Virus Res. 32(2), 97-109.
Coffin J (1985). Genome Structure (R Weiss, N Teich, H E Varmus eds) 2, 17-74.
Graf M, Bojak A, Deml L, Bieler K, Wolf H, Wagner R (2000) J. Virol. 74, 10822-826.
Harvey A J, Speksnijder G, Baugh L R, Morris J A, Ivarie R (2002) Poult. Sci. 81(2), 202-12.
Horton R M, Hunt H D, Ho S N, Pullen J K, Pease L R. (1989) Gene 77(1), 61-8.
Levy D E, Lerner R A, Wilson M C (1985). Cell 41, 289-299.
Lois c, Hong E J, Pease S, Brown E J, Baltimore D (2002) Science 295(5556), 868-72.
Martinez-Salas E (1999) Current Opinion Biotechnology 10, 458-64.
McGrew M J, Sherman A, Ellard F M, Lillico S G, Gilhooley H J, Kingsman A J, Mitrophanous K A & Sang H (2004) EMBO Reports 5(7), 728-33.
Nudler E, Avetissova E, Markovtsov V, Goldfarb A (1996) Science 273, 211-217.
Omer C A, Pogue-geile K, Guntaka R, Staskis K A, Faras A J (1983). J. Virol. 54, 889-893.
Pain B, Clark M E, Shen M, Nakazawa H, Sakurai M, Samarut J, Etches R J, (1996). Development 122(8), 2339-48.
Pfeifer A, Ikawa M, dayn Y, Verma I M (2002) PNAS 99(4), 2140-45.
Schneider R, Campbell M, Nasioulas G, Felber B K, Pavlakis G N (1997). Journal of Virology 71(7), 4892-903.
Shaw G, Kamen R (1986). Cell 46(5), 659-67.
Spies M, Bianco P R, Dillingham M S, Handa N, Baskin R J, Kowalczykowski S C (2003). Cell 114(5), 647-54.
Tran D P, Kim S J, Park N J, Jew T M, Martinson H G (2001). Molecular and Cellular Biology 21(21), 7495-508.
Trinh R, Gurbaxani B, Morrison S L, Seyfzadeh M (2004). Molecular immunology 40, 717-722.
White K A and Morris T J (1995) RNA 1, 1029-1040.
Weck, E. 1999 ‘Transgenic Animals: ‘market opportunities now a reality’ D&MD reports

1. A method of optimising an exogenous DNA sequence for expression by a suitable vector, the method comprising the steps of:
optimising the nucleotide codon usage of the exogenous DNA sequence to alter codon usage to that of the host cell type in which the exogenous DNA sequence is to be expressed,
modifying the codon optimised exogenous DNA sequence to alter any area of sequence which may prevent or down regulate expression of the exogenous DNA in the host cell, and
altering the nucleotide codon usage of the exogenous DNA sequence in order to remove all sequences implicated in the putative homologous recombination-based deletion mechanism.

2. A method as claimed in claim 1 wherein the exogenous DNA sequence encodes for a heterologous protein.

3. A method as claimed in claim 1 wherein the exogenous DNA sequence encodes for an antibody.

4. A method as claimed in claim 3 which additionally includes the step of designing a linker sequence for inclusion in the antibody coding sequence, said linker sequence having substantially all of the direct repeats removed from the DNA coding sequence, while still retaining the three direct repeats of (Gly₄Ser₁) in the primary amino acid sequence.

5. A method as claimed in claim 4 wherein the step of designing a linker sequence for inclusion in the antibody coding sequence is performed prior to the performance of step (iii).

6. A method as claimed in claim 1 wherein the sequence which may prevent or down regulate expression of the exogenous DNA sequence in the host cell is selected from the group comprising: negative elements or repeat sequences, cis-acting motifs such as splice sites, internal TATA-boxes and ribosomal entry sites.

7. (canceled)

8. A method as claimed in claim 1 wherein the vector is introduced into a transgenic expression system.

9. A method as claimed in claim 8 wherein the transgenic expression system is a transgenic avian.

10. A method as claimed in claim 9 wherein the transgenic avian is a chicken.

11. A method as claimed in claim 1 wherein the vector is a lentiviral vector.

12. A method as claimed in claim 1 wherein the vector is Equine Infectious Anaemia Virus (EIAV).

13. A linker sequence for a recombinant antibody, said linker sequence having a sequence as defined in SEQ ID NO: 1.

14. A linker sequence for a recombinant antibody, the nucleotide sequence of said linker sequence excluding the presence of short, direct repeat DNA sequences and GGC and TCC as adjacent codons.

15. A linker sequence for the expression of a recombinant antibody-based transgene, said linker sequence having a nucleotide sequence according to SEQ ID NO: 3.

16. A linker sequence for the expression of a recombinant antibody-based transgene, said linker sequence having an amino acid sequence according to SEQ ID NO: 4.

17. A method of producing a transgenic avian, the method comprising the steps of:
providing an exogenous DNA sequence which encodes for at least one heterologous protein, the expression of which is desired in the transgenic avian,
performing codon optimisation of the nucleotide sequence of the heterologous protein coding region of the exogenous DNA sequence to alter codon usage to that of the avian cell in which the heterologous protein is to be expressed,
modifying the exogenous DNA sequence to change any coding sequence regions which are predicted to prevent or down regulate gene expression in the host avian,
altering codon usage of the exogenous DNA sequence in order to remove all sequences implicated in the putative homologous recombination-based deletion mechanism,
integrating a vector comprising the exogenous DNA sequence into the genome of an avian, and
expressing said exogenous DNA sequence in order to produce the heterologous protein encoded by said sequence.

18. A method as claimed in claim 17 wherein the transgenic avian is a chicken, turkey, duck, quail, goose, ostrich, pheasant, peafowl, guinea fowl, pigeon, swan, bantam or penguin.

19. A method as claimed in claim 17 wherein the transgenic avian is a chimeric avian or a mosaic avian.

20. A method as claimed in claim 17 wherein expression of the heterologous protein is directed in a tissue specific manner.

21. A method as claimed in claim 17 wherein expression of the heterologous protein is directed to the oviduct.

22. A method as claimed in claim 17 wherein expression of the heterologous protein is included in the egg.

23. A method as claimed in claim 17 wherein expression of the heterologous protein is directed to the egg white.

24. A method of expressing an exogenous protein in an avian, said method comprising the steps of:
providing an exogenous DNA sequence encoding for at least one exogenous protein, expression of which is desired within the avian,
analysing said exogenous DNA sequence using the method according to claim 1,
expressing the exogenous DNA sequence into the genome of an avian,
obtaining the expressed exogenous protein from the avian.

25. A method of expressing a heterologous protein in the oviduct of an avian, the method comprising the steps of;
providing an exogenous DNA sequence which has been analysed using the method of claim 1 to remove or replace any areas of coding sequence which may prevent or down regulate the expression of the heterologous protein encoded by the exogenous DNA sequence,
integrating the exogenous DNA coding sequence into the genome of an avian,
expressing the exogenous DNA coding sequence by means of a promoter which is operably linked to the exogenous DNA sequence, and
obtaining the exogenous protein expressed by said transgenic avian.

26. A method as claimed in claim 25 wherein the exogenous DNA coding sequence is inserted into a viral vector backbone, with this vector being inserted into an avian cell.

27. A method as claimed in claim 1 wherein the exogenous DNA sequence analysed using the method of claim 1 is used to produce an avian egg containing at least one exogenous protein.

28. A method as claimed in claim 1 wherein the exogenous DNA sequence analysed using the method of claim 1 is used to produce a heterologous protein product, said product being the result of transcription and translation of at least part of the exogenous DNA sequence.

29. An expression vector which comprises at least one exogenous DNA sequence which has been analysed according to the method of claim 1.

30. A host cell transduced with an expression vector of claim 29.

31. A kit for the performance of the method of claim 1, said kit comprising instructions and protocols for the performance of said method.


SEQUENCE LISTING (23 sequences)

SEQ ID NO: 1 - DNA, 45 bp, Artificial
  Linker in RPAS Mouse scFV Module (Amersham Biosciences)
ggtggaggcggttcaggcggaggtggctctggcggtggcggatcg

SEQ ID NO: 2 - protein, 15 aa, Artificial
  Linker in RPAS Mouse scFV Module (Amersham Biosciences)
GGGGSGGGGSGGGGS

SEQ ID NO: 3 - DNA, 45 bp, Artificial
  Linker of the present invention
gggggagggggcagcggcggagggggatccggcggtgggggatct

SEQ ID NO: 4 - protein, 15 aa, Artificial
  Linker of the present invention
GGGGSGGGGSGGGGS

SEQ ID NO: 5 - DNA, 45 bp, Artificial
  Linker in RPAS Mouse scFV Module (Amersham Biosciences) assessed for presence of GGC and TCC as adjacent codons
gggggaggcggctccgggggaggcggctccgggggaggcggctcc

SEQ ID NO: 6 - DNA, 1500 bp, Artificial
  R24 Minibody used in construction of pRI28 and pLE38
atgaggtctttgctaatcttggtgctttgcttcctgcccctggctgctctgggggatgtgcagctggtggagtccgggggaggcctggtgcagcccggagggtcccgcaagctctcctgcgccgcctccggattcaccttcagcaacttcggaatgcactgggtgcgccaggcccccgagaaggggctggagtgggtgggatacatcagcagcggcggcagctccatcaactacgccgacaccgtgaagggccgcttcaccatctccagagacaaccccaagaacaccctgttcctgcagatgaccagcctgaggtccgaggacacagccatctactactgcaccagagggggaaccgggaccagatccctgtactacttcgactactggggccagggcgccacactgatcgtgtcctccgggggaggcggctccgggggaggcggctccgggggaggcggctccgatatccagatgacacagatcacatcctccctgtctgtgtctctgggagacagagtgatcatcagctgcagggctagccaggacatcggcaattttctgaactggtaccagcaggaaccagatggatctctgaagctgctgatctactacacatctagactgcagtccggagtgccatccaggttcagcggctgggggtctggaacagattactctctgaccattagcaacctggaggaagaggatatcgccaccttcttctgccagcagggcaagacactgccctacaccttcggaggggggaccaagctggagatcaagcgcggatccgccagacccaagtcctgcgacaagacccacacatgcccaccctgcccagcccccgagctgctggggggaccctccgtgttcctgttccccccaaagcccaaggacaccctgatgatctcccgcacccccgaggtgacatgcgtggtggtggacgtgagccacgaggaccccgaggtgaagttcaactggtacgtggacggcgtggaggtgcacaacgccaagacaaagccccgcgaggagcagtacaacagcacctaccgcgtggtgagcgtgctgaccgtgctgcaccaggactggctgaacggcaaggagtacaagtgcaaggtgtccaacaaggccctgccagcccccatcgagaagaccatctccaaggccaaggggcagccccgcgagccacaggtgtacaccctgcccccatcccgcgaggagatgaccaagaaccaggtgagcctgacctgcctggtgaagggcttctaccccagcgacatcgccgtggagtgggagagcaacgggcagcccgagaacaactacaagaccaccccccccgtgctggactccgacggctccttcttcctgtacagcaagctgaccgtggacaagagcaggtggcagcaggggaacgtgttctcctgctccgtgatgcacgaggccctgcacaaccactacacccagaagagcctctccctgtcccccggcaagtgataa

SEQ ID NO: 7 - protein, 498 aa, Artificial
  Synthetic Construct
MRSLLILVLCFLPLAALGDVQLVESGGGLVQPGGSRKLSCAASGFTFSNFGMHWVRQAPEKGLEWVGYISSGGSSINYADTVKGRFTISRDNPKNTLFLQMTSLRSEDTAIYYCTRGGTGTRSLYYFDYWGQGATLIVSSGGGGSGGGGSGGGGSDIQMTQITSSLSVSLGDRVIISCRASQDIGNFLNWYQQEPDGSLKLLIYYTSRLQSGVPSRFSGWGSGTDYSLTISNLEEEDIATFFCQQGKTLPYTFGGGTKLEIKRGSARPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 7907 bp, Artificial
  Lentiviral vector genome plasmid pRI28
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcattgataccaactttgtaaaagaaaaggactggcagctgagggattgtcattccattgctggaagattgtaactcagacgctgtcaggacaagaaagagaggcctttgaaagaacattggtgggcaatttctgctgtaaagattgggcctccagattaataattgtagtagattggaaaggcatcattccagctcctaagagcgaaatattgaaaagaagactgctaataaaaagcagtctgagccctctgaagaatatctctagaactagtggatcccccgggccaaaacctagcgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggccgcgaattcaaaagctagagtcgactctagggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagctagtcgactttaagcttctcgagggcgcgccttcgaacacgggcaacgccaccatgaggtctttgctaatcttggtgctttgcttcctgcccctggctgctctgggggatgtgcagctggtggagtccgggggaggcctggtgcagcccggagggtcccgcaagctctcctgcgccgcctccggattcaccttcagcaacttcggaatgcactgggtgcgccaggcccccgagaaggggctggagtgggtgggatacatcagcagcggcggcagctccatcaactacgccgacaccgtgaagggccgcttcaccatctccagagacaaccccaagaacaccctgttcctgcagatgaccagcctgaggtccgaggacacagccatctactactgcaccagagggggaaccgggaccagatccctgtactacttcgactactggggccagggcgccacactgatcgtgtcctccgggggaggcggctccgggggaggcggctccgggggaggcggctccgatatccagatgacacagatcacatcctccctgtctgtgtctctgggagacagagtgatcatcagctgcagggctagccaggacatcggcaattttctgaactggtaccagcaggaaccagatggatctctgaagctgctgatctactacacatctagactgcagtccggagtgccatccaggttcagcggctgggggtctggaacagattactctctgaccattagcaacctggaggaagaggatatcgccaccttcttctgccagcagggcaagacactgccctacaccttcggaggggggaccaagctggagatcaagcgcggatccgccagacccaagtcctgcgacaagacccacacatgcccaccctgcccagcccccgagctgctggggggaccctccgtgttcctgttccccccaaagcccaaggacaccctgatgatctcccgcacccccgaggtgacatgcgtggtggtggacgtgagccacgaggaccccgaggtgaagttcaactggtacgtggacggcgtggaggtgcacaacgccaagacaaagccccgcgaggagcagtacaacagcacctaccgcgtggtgagcgtgctgaccgtgctgcaccaggactggctgaacggcaaggagtacaagtgcaaggtgtccaacaaggccctgccagcccccatcgagaagaccatctccaaggccaaggggcagccccgcgagccacaggtgtacaccctgcccccatcccgcgaggagatgaccaagaaccaggtgagcctgacctgcctggtgaagggcttctaccccagcgacatcgccgtggagtgggagagcaacgggcagcccgagaacaactacaagaccaccccccccgtgctggactccgacggctccttcttcctgtacagcaagctgaccgtggacaagagcaggtggcagcaggggaacgtgttctcctgctccgtgatgcacgaggccctgcacaaccactacacccagaagagcctctccctgtcccccggcaagtgataagtccacgtgcgtacgtcgcgaaccggttgatcattaattaagggccctagcttatcgataccgtcgaattggaagagctttaaatcctggcacatctcatgtatcaatgcctcagtatgtttagaaaaacaaggggggaactgtggggtttttatgaggggttttatacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccgggagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 9 - DNA, 1866 bp, Artificial
  3′ end of pLE38 genome encompassing R24 coding sequence
atgaggtctttgctaatcttggtgctttgcttcctgcccctggctgctctgggggatgtgcagctggtggagtccgggggaggcctggtgcagcccggagggtcccgcaagctctcctgcgccgcctccggattcaccttcagcaacttcggaatgcactgggtgcgccaggcccccgagaaggggctggagtgggtgggatacatcagcagcggcggcagctccatcaactacgccgacaccgtgaagggccgcttcaccatctccagagacaaccccaagaacaccctgttcctgcagatgaccagcctgaggtccgaggacacagccatctactactgcaccagagggggaaccgggaccagatccctgtactacttcgactactggggccagggcgccacactgatcgtgtcctccgggggaggcggctccgggggaggcggctccgggggaggcggctccgatatccagatgacacagatcacatcctccctgtctgtgtctctgggagacagagtgatcatcagctgcagggctagccaggacatcggcaattttctgaactggtaccagcaggaaccagatggatctctgaagctgctgatctactacacatctagactgcagtccggagtgccatccaggttcagcggctgggggtctggaacagattactctctgaccattagcaacctggaggaagaggatatcgccaccttcttctgccagcagggcaagacactgccctacaccttcggaggggggaccaagctggagatcaagcgcggatccgccagacccaagtcctgcgacaagacccacacatgcccaccctgcccagcccccgagctgctggggggaccctccgtgttcctgttccccccaaagcccaaggacaccctgatgatctcccgcacccccgaggtgacatgcgtggtggtggacgtgagccacgaggaccccgaggtgaagttcaactggtacgtggacggcgtggaggtgcacaacgccaagacaaagccccgcgaggagcagtacaacagcacctaccgcgtggtgagcgtgctgaccgtgctgcaccaggactggctgaacggcaaggagtacaagtgcaaggtgtccaacaaggccctgccagcccccatcgagaagaccatctccaaggccaaggggcagccccgcgagccacaggtgtacaccctgcccccatcccgcgaggagatgaccaagaaccaggtgagcctgacctgcctggtgaagggcttctaccccagcgacatcgccgtggagtgggagagcaacgggcagcccgagaacaactacaagaccaccccccccgtgctggactccgacggctccttcttcctgtacagcaagctgaccgtggacaagagcaggtggcagcaggggaacgtgttctcctgctccgtgatgcacgaggccctgcacaaccactacacccagaagagcctctccctgtcccccggcaagtgataagtccacggggcatcactagtgaattcgcggccgcctgcaggtcgaccatatgggagagctcccaacgcgcgcgccttcgaacacgtgcgtacgtcgcgaaccggttgatcattaattaagggccctagcttatcgataccgtcgaattggaagagctttaaatcctggcacatctcatgtatcaatgcctcagtatgtttagaaaaacaaggggggaactgtggggtttttatgaggggttttatacaattgggcactcagattctgcggtctgagtcccttctctgctgggctgaaaaggcctttgtaataaatataattctctactcagtccctgttctagtttgtctgttcgagatcctacag

SEQ ID NO: 10 - protein, 498 aa, Artificial; identical to SEQ ID NO: 7
  Synthetic Construct

SEQ ID NO: 11 - protein, 129 aa, Artificial
  R24 minibody VH domain
LPLAALGDVQLVESGGGLVQPGGSRKLSCAASGFTFSNFGMHWVRQAPEKGLEWVGYISSGGSSINYADTVKGRTFISRDNPKNTLFLQMTSLRSEDTAIYYCTRGGTGTRSLYYFDYWGQGATLIVSS

SEQ ID NO: 12 - protein, 128 aa, Artificial
  R24 minibody VL domain
GGGGSGGGGSGGGGSDIQMTQITSSLSVSLGDRVIISCRASQDIGNFLNWYQQEPDGSLKLLIYYTSRLQSGVPSRFSGWGSGTDYSLTISNLEEEDIATFFCQQGKTLPYTFGGGTKLEIKRGSARP

SEQ ID NO: 13 - DNA, 510 bp, Artificial
  BspEI fragment inserted into pLE8 during the lt1 repair process
tccggattcaccttcagcaacttcggcatgcactgggtgagacaggcccccgagaaggggctggagtgggtgggatacatcagcagcggaggcagcagcatcaactacgccgacaccgtgaagggccgctttaccatctcccgcgacaaccccaagaacaccctgttcctgcagatgaccagcctgagaagcgaagataccgccatctactactgcaccagggggggaaccgggaccagatccctgtactactttgactactggggccagggagccacactcattgtgtcctccgggggagggggcagcggcggagggggatccggcggtgggggatctgacatccagatgactcagattacatcctccctgagcgtgtccctgggcgacagagtgattatcagctgcagggcttcccaggacatcggcaattttctgaattggtatcagcaggagcccgacggatccctgaaactgctgatctactacacaagcagactgcagtccgga

SEQ ID NO: 14 - DNA, 45 bp, Artificial; identical to SEQ ID NO: 5
  Original linker present in standard R24 minibody

SEQ ID NO: 15 - protein, 15 aa, Artificial
  Synthetic Construct
GGGGSGGGGSGGGGS

SEQ ID NO: 16 - DNA, 45 bp, Artificial; identical to SEQ ID NO: 3
  Modified linker present in repaired R24 minibody

SEQ ID NO: 17 - protein, 15 aa, Artificial
  Synthetic Construct
GGGGSGGGGSGGGGS

SEQ ID NO: 18 - protein, 503 aa, Artificial
  Primary amino acid sequence of optimised anti-CD55 minibody
MRSLLILVLCFLPLAALGAATMAQVQLQESGAELARPGASVKMSCKASGYAFTTYTMHWVKQRPGQGLEWIGYINPTNDYTNYHQNFKDKATLTADKSSSTAYMQLNSLTSEDSAVYYCSRRGVLNKRYYALDYWGQGTTVTVSSGGGGSGGGGSGGGGSDIVLTQTTKFLLVSAGDRVTITCKASQSVSNDVAWYQQKPGQSPKLLIYFASSRFTGVPDCFIGSGYGTDFTFTITTVQAEDLAVYFCQQDYSSPLTFGAGTKPELKRGSARPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1514 bp, Artificial
  DNA sequence of optimised anti-CD55 minibody
atgaggagcctgctgattctggtgctgtgcttcctcccactggctgctctgggagccgccaccatggcccaagtgcagctgcaggagagcggggctgaactggcaagacctggggccagcgtgaagatgtcctgcaaggctagcggctacgcctttactacctacaccatgcactgggtgaaacagaggcctggacagggcctggaatggatcggatacatcaaccctaccaacgattacactaactaccaccagaacttcaaagacaaggccacactgactgcagacaaatcctccagcacagcctacatgcagctgaacagcctgacaagcgaggatagcgcagtgtactactgcagcagaagaggcgtgctgaacaaacgctactacgctctggactactggggccaggggaccaccgtgaccgtgtccagcgggggagggggcagcggcggagggggatccggcggtgggggatctgacatcgtgctgacccagactacaaaattcctgctggtgagcgcaggagaccgcgtgaccatcacctgcaaggccagccagagcgtgagcaacgatgtggcttggtatcagcagaagccagggcagagccctaaactgctgatttactttgcatccagccgcttcactggagtgcctgattgcttcatcggcagcggatacgggaccgatttcactttcaccatcaccactgtgcaggctgaggacctggccgtgtacttctgccagcaggattacagcagccccctgaccttcggcgctgggaccaagcccgagctgaaacggggatccgccagacccaagtcctgcgacaagacccacacatgcccaccctgcccagcccccgagctgctggggggaccctccgtgttcctgttccccccaaagcccaaggacaccctgatgatctcccgcacccccgaagtgacatgcgtggtggtggacgtgagccacgaggatcccgaagtgaagttcaactggtacgtggacggcgtggaagtgcacaacgccaagacaaagccccgcgaggagcagtacaacagcacctaccgcgtggtgagcgtgctgaccgtgctgcaccaggactggctgaacggaaaggagtacaagtgcaaagtgtccaacaaggccctgccagctcccatcgagaaaaccatctccaaggccaaggggcagcccagggagccacaagtgtacaccctgccaccaagccgcgaggagatgaccaagaaccaagtgagcctgacctgcctggtgaaaggcttctaccccagcgacatcgccgtggagtgggagagcaacgggcagcccgagaacaactacaagaccacaccacccgtgctggactccgacggaagcttcttcctgtactccaaactgaccgtggacaagagccgctggcagcaggggaacgtgttctcctgctccgtgatgcacgaggccctgcacaaccactacacccagaagagcctgtccctgtcccccggcaagtgata

SEQ ID NO: 20 - protein, 490 aa, Artificial
  Primary amino acid sequence of heavy chain of anti-CD55 antibody
MRSLLILVLCFLPLAALGQVQLEESGAELARPGASVKMSCKASGYAFTTYTMHWVKQRPGQGLEWIGYINPTNDYTNYHQNFKDKATLTADKSSSTAYMQLNSLTSEDSAVYYCSRRGVLNKRYYALDYWGQGTSVTVSSAKTTPPSVYPLARSSQSNDIPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 248 aa, Artificial
  Primary amino acid sequence of light chain of anti-CD55 antibody
MRSLLILVLCFLPLAALGSIVMTQTPKFLLVSAGDRVTITCKASQSVSNDVAWYQQKPGQSPKLLIYFASSRFTGVPDRFIGSGYGTDFTFTITTVQAEDLAVYFCQQDYSSPLTFGAGTKPELKRADAAPTVSACTNHDIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 1120 bp, Artificial
  3′ end of pLE119 genome encompassing anti-CD55 coding sequence
atgaggagcctgctgattctggtgctgtgcttcctgcctctggccgccctgggcagcatcgtgatgacccagacccccaagttcctgctggtgtccgccggagatagagtgaccatcacctgcaaggccagccagagcgtgtccaacgatgtggcctggtatcagcagaagcccggccagagccccaagctgctcatctacttcgccagcagcagattcacaggcgtgcccgacagattcatcggcagcggctacggcaccgatttcaccttcaccatcaccacagtgcaggccgaggatctggccgtgtacttttgccagcaggactacagcagcccactgacattcggcgctggcacaaagcccgagctgaagagagccgacgccgctcccacagtgagcgcctgcaccaaccacgatatcagaaccgtggccgctcccagcgtgttcatcttcccccccagcgatgagcagctgaagagcggcaccgccagcgttgtgtgcctgctgaacaacttctacccccgcgaggccaaagtgcagtggaaagtggacaacgccctgcagagcggcaacagccaggagagcgtgacagagcaggacagcaaggactccacctacagcctgagcagcaccctgaccctgagcaaggccgactacgagaagcacaaagtgtacgcctgcgaagtgacccaccagggactgagcagccccgtgacaaagagcttcaaccgcggcgagtgctgatagtctagacccggggcatcactagtgaattcgcggccgcctgcaggtcgaccatatgggagagctcccaacgcgcgcgccttcgaacacgtgcgtacgtcgcgaaccggttgatcattaattaagggccctagcttatcgataccgtcgaattggaagagctttaaatcctggcacatctcatgtatcaatgcctcagtatgtttagaaaaacaaggggggaactgtggggtttttatgaggggttttatacaattgggcactcagattctgcggtctgagtcccttctctgctgggctgaaaaggcctttgtaataaatataattctctactcagtccctgtctctagtttgtctgttcgagatcctacag

SEQ ID NO: 23 - protein, 248 aa, Artificial; identical to SEQ ID NO: 21
  Synthetic Construct